(19) United States
(12) Patent Application Publication
(10) Pub. No.: US 2006/0205688 A1
(43) Pub. Date: Sep. 14, 2006

(54) Method of modulating epithelial cell activity by modulating the functional levels of sphingosine kinase

(75) Inventors: Jennifer Ruth Gamble, Stirling (AU); Matthew Vadas, Stirling (AU); Stuart Pitson, Norwood (AU); Pu Xia, Magill (AU); Vidya Limaye, St. Peters (AU)

Correspondence Address: SEED INTELLECTUAL PROPERTY LAW GROUP PLLC, 701 FIFTH AVE, SUITE 6300, SEATTLE, WA, 98104-7092, US

(73) Assignee: Medvet Science Pty. Ltd., Stepney (AU)

(21) Appl. No.: 10/531,626

(22) PCT Filed: Oct. 14, 2003
(86) PCT No.: PCT/AU03/01356

§ 371 Date: Mar. 30, 2006

(30) Foreign Application Priority Data
Oct. 14, 2002 (AU) 2002952032
Apr. 30, 2003 (AU) 2003902047

(51) Int. Cl. A61K 48/00

(52) U.S. Cl. 514/44

(57) ABSTRACT

The present invention relates generally to a method of modulating endothelial cell functional characteristics and to agents useful for same. More particularly, the present invention relates to a method of modulating vascular endothelial cell pro-inflammatory and angiogenic phenotypes by modulating the functional levels of intracellular sphingosine kinase. The method of the present invention is useful, inter alia, in relation to the treatment and/or prophylaxis of conditions which are characterised by inadequate endothelial cell functioning and may include conditions such as vascular engraftment, organ transplantation or wound healing or conditions which are characterised by an aberrant endothelial cell inflammatory or angiogenic phenotype. Further, the method of the present invention facilitates the development of agents, such as functionally manipulated endothelial cell populations, for a range of therapeutic and/or prophylactic uses.

### FIELD OF THE INVENTION

The present invention relates generally to a method of modulating endothelial cell functional characteristics and to agents useful for same. More particularly, the present invention relates to a method of modulating vascular endothelial cell pro-inflammatory and angiogenic phenotypes by modulating the functional levels of intracellular sphingosine kinase. The method of the present invention is useful, inter alia, in relation to the treatment and/or prophylaxis of conditions which are characterised by inadequate endothelial cell functioning and may include conditions such as vascular engraftment, organ transplantation or wound healing or conditions which are characterised by an aberrant endothelial cell inflammatory or angiogenic phenotype. Further, the method of the present invention facilitates the development of agents, such as functionally manipulated endothelial cell populations, for a range of therapeutic and/or prophylactic uses.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that the prior art forms part of the common general knowledge.

The survival and proliferation of cells is dependent upon an adequate supply of oxygen and nutrients and the removal of toxins. Angiogenesis is the name given to the development of new capillaries from pre-existing blood vessels. In order for stimulated endothelial cells to form a new blood vessel, they must proliferate, migrate and invade the surrounding tissue.

In adult mammals, the vasculature is quiescent, except during the physiological cycle of reproduction or in the case of wound healing. Further, additional requirements in terms of oxygen or nutrients will usually result in sprouting of new capillaries from pre-existing vessels. Local hyper-vascularisation is thought to result from release by tissues of soluble media which has induced the switch of the quiescent endothelial cell phenotype to the activated one, in order for endothelial cells to be able to respond to mitogenic signals. The release of mitogenic growth factors allows the activation of the receptors that signal for cell migration, proliferation and differentiation into new capillaries and thereby switches the activated phenotype to an angiogenic phenotype.

There is an ongoing need to develop methods for facilitating angiogenesis, such as in the context of vascularisation of grafts or wound healing. In terms of working with and manipulating endothelial cells, there are certain inherent functional limitations such as the requirement for attachment and cell spreading mediated anti-apoptotic signals in order to maintain endothelial cell viability. Further, activation of endothelial cell differentiation generally results in loss of the haematoprogenitor cell marker CD34. This irreversibly alters the phenotype of the activated endothelial cells.

In light of the significant interest in promoting angiogenesis in both the in vitro and in vivo environments, there is a need to develop means of both facilitating the maintenance of optimal endothelial cell phenotypes and promoting optimal endothelial cell growth. In work leading up to the present invention, it has been determined that over expression of the human sphingosine kinase gene in human endothelial cells results in enhanced endothelial cell proliferation and cell survival relative to normal cells. Further, sphingosine kinase over expression has been determined to maintain the endothelial cell haematoprogenitor phenotype, as characterised by the expression of CD34, despite the induction of endothelial cell proliferation. Still further, sphingosine kinase over-expression induces endothelial cell inflammatory and angiogenic phenotypes. Accordingly, there is now provided a means of facilitating the therapeutic manipulation of endothelial cell proliferation and differentiation based on modulation of intracellular sphingosine kinase levels.

### SUMMARY OF THE INVENTION

Throughout this specification and the claims which follow, unless the context requires otherwise, the word “comprise”, and or variations such as “comprises” or “comprising”, will be understood to imply the inclusion of a stated integer or step or group of integers or steps but not the exclusion of any other integer or step or group of integers or steps.

One aspect of the present invention is directed to a method of modulating one or more endothelial cell functional characteristics, said method comprising modulating the functional level of sphingosine kinase wherein inducing over-expression of said sphingosine kinase level modulates one or more of the functional characteristics of said endothelial cell.

In another aspect there is provided a method of modulating one or more vascular endothelial cell functional characteristics, said method comprising modulating the functional level of sphingosine kinase wherein inducing over-expression of said sphingosine kinase level modulates one or more of the functional characteristics of said vascular endothelial cell.

In yet another aspect there is provided the method of modulating one or more CD34⁺ endothelial cell functional characteristics, said method comprising modulating the functional level of sphingosine kinase wherein inducing over-expression of said sphingosine kinase level modulates one or more of the functional characteristics of said CD34⁺ endothelial cell.

The present invention also provides a method of modulating one or more endothelial cell functional characteristics, said method comprising modulating the functional level of sphingosine kinase wherein up-regulating said sphingosine kinase level modulates one or more of the functional characteristics of said endothelial cell relative to normal endothelial cell functional characteristics.

Preferably, said endothelial cell is a vascular endothelial cell.

In still another aspect there is provided a method of modulating vascular endothelial cell proliferation, said method comprising modulating the functional level of sphingosine kinase wherein inducing over-expression of said sphingosine kinase level enhances the proliferation of said endothelial cell relative to normal endothelial cell proliferation.

In still yet another aspect there is provided a method of modulating vascular endothelial viability, said method comprising modulating the functional level of sphingosine kinase wherein inducing over-expression of said sphingosine kinase level enhances the viability of said vascular endothelial cell relative to normal endothelial cell viability.

In yet still another aspect there is provided a method of modulating the CD34⁺ endothelial cell progenitor phenotype, said method comprising modulating the functional level of sphingosine kinase wherein inducing over-expression of said sphingosine kinase level maintains the CD34⁺ endothelial cell progenitor phenotype.

A further aspect of the present invention is directed to a method of modulating one or more endothelial cell functional characteristics in a mammal, said method comprising modulating the functional level of sphingosine kinase wherein inducing over-expression of said sphingosine kinase level modulates one or more of the functional characteristics of said endothelial cell.

In another further aspect said method is directed to modulating one or more vascular endothelial cell functional characteristics in a mammal, said method comprising modulating the functional level of sphingosine kinase in said mammal wherein inducing over-expression of said sphingosine kinase level modulates one or more of the functional characteristics of said endothelial cell.

The present invention also provides a method of modulating one or more endothelial cell functional characteristics, said method comprising modulating the functional level of sphingosine kinase wherein up-regulating said sphingosine kinase level modulates one or more of the functional characteristics of said endothelial cell relative to normal endothelial cell functional characteristics.

In yet another further aspect there is provide a method of modulating vascular endothelial cell proliferation in a mammal, said method comprising modulating the functional level of sphingosine kinase in said mammal wherein inducing over-expression of said sphingosine kinase level enhances the proliferation of said endothelial cell relative to normal endothelial cell proliferation.

In still another further aspect there is provided a method of modulating vascular endothelial cell viability in a mammal, said method comprising modulating the functional level of sphingosine kinase in said mammal wherein inducing over-expression of said sphingosine kinase level enhances the viability of said vascular endothelial cell relative to normal endothelial cell viability.

In yet another aspect there is provided a method of modulating the CD34⁺ endothelial cell progenitor phenotype in a mammal, said method comprising modulating the functional level of said sphingosine kinase in said mammal wherein inducing over-expression of said sphingosine kinase level maintains the CD34⁺ endothelial cell progenitor phenotype.

Another aspect of the present invention contemplates a method for the treatment and/or prophylaxis of a condition characterised by aberrant or otherwise unwanted endothelial cell functioning in a mammal, said method comprising modulating the functional level of sphingosine kinase in said mammal wherein inducing over-expression of said sphingosine kinase level modulates one or more functional characteristics of said endothelial cells.

Yet another aspect of the present invention provides a method for the treatment and/or prophylaxis of a condition characterised by aberrant or otherwise unwanted vascular endothelial cell functioning in a mammal, said method comprising modulating the functional level of sphingosine kinase in said mammal wherein inducing over-expression of said sphingosine kinase level modulates one or more functional characteristics of said endothelial cells.

In still another aspect there is provided a method for the treatment and/or prophylaxis of a condition characterised by aberrant or otherwise unwanted vascular endothelial cell functioning in a mammal, said method comprising administering to said mammal an effective amount of an agent for a time and under conditions sufficient to modulate the functional level of sphingosine kinase.

Another aspect of the present invention relates to the use of an agent capable of modulating the functional level of sphingosine kinase in the manufacture of a medicament for the modulation of one or more endothelial cell functional characteristics in a mammal wherein inducing over-expression of said sphingosine kinase level modulates one or more of the functional characteristics of said endothelial cells.

In another aspect, the present invention relates to the use of sphingosine kinase or a nucleic acid encoding sphingosine kinase in the manufacture of a medicament for the modulation of one or more endothelial cell functional characteristics in a mammal wherein inducing over-expression of said sphingosine kinase level modulates one or more of the functional characteristics of said endothelial cells.

In yet another further aspect, the present invention contemplates a pharmaceutical composition comprising the modulatory agent as hereinbefore defined and one or more pharmaceutically acceptable carriers and/or diluents.

Still another aspect of the present invention is directed to a method of generating an endothelial cell, which endothelial cell is characterised by the modulation of one or more functional characteristics relative to normal endothelial cell functional characteristics, said method comprising inducing over-expression of the functional level of sphingosine kinase in said cell.

Yet another aspect of the present invention is directed to the endothelial cells which are generated in accordance with the methods defined herein.

Still yet another aspect of the present invention is directed to the use of endothelial cells developed in accordance with the method defined herein in the treatment and/or prophylaxis of conditions characterised by inadequate endothelial cell functioning.

### BRIEF DESCRIPTION OF THE DRAWINGS

FIG. 1 is an image showing the survival of HUVEC over-expressing SK or EV as reflected by the optical density, in the absence of FCS (a) and in the absence of both FCS and attachment to extracellular matrix (b). (a) shows the pooled data of 43 observations derived from 9 separate experiments, (b) shows the pooled data of 10 observations from two separate experiments, normalized to Day 0=1. *p<0.001 compared with corresponding vector at Day 0. Bars represent 95% confidence intervals. (c) shows (by Western blot) cyclin D1 and cyclin E expression in cells over-expressing SK and control (EV) under basal conditions (24 hours in endothelial basal medium supplemented with 0.5% FCS without growth factors) and in response to 24 hours of stimulation with growth factors). The loading control Flt-1 (VEGF-RI) is indicated.

FIG. 2 is an image showing a DAPI stain performed on cells over-expressing SK and control (EV) in culture medium supplemented with 20% FCS (a) or serum free medium (b). Apoptotic cells show intense nuclear staining of DAPI.

FIG. 3 is a graphical representation of caspase-3 activity in cells over-expressing SK and EV control, measured under basal culture conditions (a), or after 24 hours of serum deprivation (b). The figure shows the pooled data from five separate endothelial cell lines, normalized to EV=1 (a) or EV=10 (b). *p<0.05 compared with EV. Bars represent 95% confidence intervals.

FIG. 4 is an image showing Western blot the phosphorylation of Akt (p-Akt) in cells overexpressing SK and control, under basal conditions and in response to six hours of serum deprivation (SF). Fig Xb shows the pooled data from five separate endothelial cell lines, *p 0.05 SK compared with EV in serum free conditions. Bars represent SEM.

FIG. 5 is a graphical representation of the effect of inhibiting the PI-3K pathway with 10 mM LY294002 (LY), or the MAPK pathway with 20 mM U0126 (UO) or 20 mM PD98059 (PD) on cell survival of HUVEC over-expressing SK (dense dots) or EV (sparse dots). A vehicle control of equivalent concentration of DMSO is indicated. The figure shows the pooled data of 8 observations from two separate experiments, adjusted to Day 0=1. Bars represent 95% confidence intervals. *p<0.001 compared with corresponding untreated cells over-expressing SK or EV at Day 2.

FIG. 6 is an image showing the effect of over-expression of SK on PECAM-1. Cell surface expression of PECAM-1 as indicated by the median fluorescence intensity (MFI) in cells over-expressing SK and EV control is indicated in (a). The figure shows the pooled data from three separate experiments, normalized to EV. *p<0.001 SK compared with EV. Bars represent 95% confidence intervals. (b) shows a Western blot for PECAM-1 and b-catenin expression in these cells. (c) shows PECAM-1 phosphorylation in cells over-expressing SK and control (EV). (d) shows the cell surface expression of VE cadherin and represents the pooled data from three separate experiments.

FIG. 7 is a graphical representation showing the permeability (normalized to time=0) of cells over-expressing SK and EV to FITC-dextran, across different time points under basal conditions (a) or in response to thrombin stimulation (0.2 units/ml) (b). (b) shows a comparison of permeability of EV and SK in response to treatment with thrombin, *p<0.001 SK compared with EV under basal conditions across all time points. The figure shows the pooled data of 7 observations from 3 separate experiments. Bars represent 95% confidence intervals.

FIG. 8 is a graphical representation of the effect of altering PECAM-1 signalling on cell survival of HUVEC over-expressing SK (dense dots) or EV control (sparse dots) in suspension (a) and in serum free conditions (b). The effect on cell survival of 20 mg/ml rabbit polyclonal anti-PECAM-1 antibody (RP), 20 mg/ml normal rabbit serum (NRS), and a monoclonal antibody directed to VE cadherin (55-7H1) at 20 mg/ml is shown. The figure shows the pooled data of ten observations from two separate experiments, normalized to Day 0=1. Bars represent 95% confidence intervals. *p<0.001 compared with untreated vector at Day 2.

FIG. 9 is a graphical representation of the effect of PECAM-1 signalling on the activation of the PI-3K/Akt pathway in cells over-expressing SK (dense dots) and EV (sparse dots). (a) shows a Western blot measuring phosphorylated Akt (p-Akt) and total Akt in basal conditions after 6 hours of serum deprivation. The effect of 20mg/mL of rabbit polyclonal anti-PECAM antibody (RP), and 20 mg/mL normal rabbit serum (NRS) is shown. (b) shows the pooled data of the quantitation of phosphorylated Akt from four separate experiments performed as in (a). Bars represent SEM. *p<0.05 of untreated SK versus untreated EV in serum free conditions, and SK treated with RP compared with untreated SK in serum free conditions.

FIG. 10 is a graphical representation of the effect of inhibiting GPCR with pertussis toxin (50 ng/ml) on cell survival in HUVEC over-expressing SK (dense dots) or EV (sparse dots). (a) shows the pooled data of eight observations from two separate experiments, normalized to Day 0=1. *p<0.05 compared with untreated SK at Day 2. Bars represent 95% confidence intervals. (b) shows the pooled data of six observations from two separate experiments, bars represent SEM. *p<0.05 compared with untreated SK.

FIG. 11 is a graphical representation demonstrating basal (a,b) and TNFα-stimulated adhesion molecule expression (c-f) for cells over-expressing SK, G82D and control (EV) achieved by infection with retrovirus (a,c,e) or adenovirus (b,d,f). VCAM-1 expression is given in a-d, and E Selectin expression in e,f. Results are normalized to EV=1 for basal, and EV=10 for stimulated expression, and bars represent 95% confidence intervals. Fig a-f show the pooled data from 3,6,4,5,4, and 6 separate experiments respectively, using different isolates of endothelial cells. *p<0.05 compared with EV.

FIG. 12 is a graphical representation demonstrating the response of VCAM-1 (a) and E Selectin (b) to very low doses of stimulation with TNFα (0.004ng/ml) for four hours in cells infected with adenovirus. The figure shows the data from a single experiment which is representative of two separate experiments in which the same trend was observed.

FIG. 13 is a graphical representation demonstrating the effect of 18 hours of treatment with 50 ng/ml pertussis toxin (PTx) on basal (a,b) and TNFα-stimulated (c,d) VCAM-1 (a,c) and E Selectin (b,d) expression, as reflected by the median fluorescence intensity (MFI) in cells over-expressing SK and control (EV). The figure shows the data from a single experiment which is representative of two separate experiments using different endothelial cell isolates.

FIG. 14 is a graphical representation demonstrating the adhesion molecule response to stimulation with SIP 5 μM for four hours in cells over-expressing SK and EV. VCAM-1 expression is shown in (a) and E Selectin expression in (b). The figure shows the pooled data from two separate experiments, and bars represent SEM. *p<0.05 compared with untreated vector by Student's t-Test.

FIG. 15 is an image (at 80× magnification) of neutrophil adhesion to endothelial cells over-expressing EV (a,d), SK (b,e) and G82D (c,f) in the basal state (a-c) and when stimulated for four hours with 0.04 ng/mL TNFα (d-f). The white arrow indicates an adherent neutrophil. The figure shows results from one experiment which is representative of two separate experiments.

FIG. 16 is a graphical representation of the number of adherent neutrophils per 100 endothelial cells, as determined from the pooled data of ten separate microscopic fields obtained from two separate experiments. Bars represent SEM. *p<0.05 compared with corresponding EV, **p<0.001 compared with corresponding EV by Student's t-Test.

FIG. 17 is an image of tube formation by cells over-expressing SK and control (EV) in Matrigel at 30 minutes (A) and at one hour (B).

### DETAILED DESCRIPTION OF THE INVENTION

The present invention is predicated, in part, on the determination that endothelial cell functional characteristics can be modulated, relative to that of normal endothelial cells, by over expressing sphingosine kinase. Specifically, it has been determined that over expressing sphingosine kinase facilitates enhanced cell proliferation and cell survival in the absence of normal anti apoptotic signals. Further, to the extent that the method of the present invention is applied to CD34 expressing endothelial cells, their progenitor-like properties can be maintained despite the onset of proliferation. Still further, endothelial cell sphingosine kinase over-expression induces an endothelial cell pro-inflammatory and angiogenic phenotype. Accordingly, the method of the present invention now permits the rational design of therapeutic and/or prophylactic methods for treating conditions characterised by inadequate endothelial cell functioning or for otherwise facilitating endothelial expansion either in vitro or in vivo. The determinations detailed herein also facilitate the development of both cellular and non-cellular agents for use in the context of treating the conditions detailed above or otherwise seeding and/or expanding an endothelial cell population.

Accordingly, one aspect of the present invention is directed to a method of modulating one or more endothelial cell functional characteristics, said method comprising modulating the functional level of sphingosine kinase wherein inducing over-expression of said sphingosine kinase level modulates one or more of the functional characteristics of said endothelial cell.

Reference to “endothelial cell” should be understood as a reference to the endothelial cells which line the blood vessels, lymphatics or other serous cavities such as fluid filled cavities. The phrase “endothelial cells” should also be understood as a reference to cells which exhibit one or more of the morphology, phenotype and/or functional activity of endothelial cells and is also a reference to mutants or variants thereof. “Variants” include, but are not limited to, cells exhibiting some but not all of the morphological or phenotypic features or functional activities of endothelial cells at any differentiative stage of development. “Mutants” include, but are not limited to, endothelial cells which have been naturally or non-naturally modified such as cells which are genetically modified. It should also be understood that the endothelial cells of the present invention may be at any differentiative stage of development. Accordingly, the cells may be immature and therefore functionally incompetent in the absence of further differentiation, such as CD34⁺ progenitor cells. In this regard, highly immature cells such as stem cells, which retain the capacity to differentiate into endothelial cells, should nevertheless be understood to satisfy the definition of “endothelial cell” as utilised herein due to their capacity to differentiate into endothelial cells under appropriate conditions. Preferably, the subject endothelial cell is a vascular endothelial cell and even more preferably a CD34⁺ endothelial cell.

Accordingly, there is more particularly provided a method of modulating one or more vascular endothelial cell functional characteristics, said method comprising modulating the functional level of sphingosine kinase wherein inducing over-expression of said sphingosine kinase level modulates one or more of the functional characteristics of said vascular endothelial cell.

Still more particularly, there is provided the method of modulating one or more CD34⁺ endothelial cell functional characteristics, said method comprising modulating the functional level of sphingosine kinase wherein inducing over-expression of said sphingosine kinase level modulates one or more of the functional characteristics of said CD34⁺ endothelial cell.

Reference to endothelial cell “functional characteristics” should be understood as reference to any one or more of the functional characteristics which an endothelial cell is capable of exhibiting. This includes, for example, proliferation, differentiation, migration, maintenance of viability in either a quiescent or active state, cell surface molecule expression, sensitization to cytokine stimulation, modulating of pro-inflammatory cytokine effects, modulated capacity to bind neutrophils and modulated inflammatory and/or angiogenic phenotype. In the context of the present invention, it has been determined that over-expression of intracellular sphingosine kinase can induce modulation of one or more endothelial cell functional characteristics. In this regard, it has been determined that in addition to modulating the normal range and degree of endothelial cell functional characteristics, the subject modulation extends to inducing functional characteristics which are not generally inducible under normal physiological conditions such as enhanced proliferative and cell survival characteristics and altered differentiation (this latter form of modulation is herein referred to as modulation of endothelial cell functional characteristics “relative to normal endothelial cell functional characteristics”). By “normal” is meant the characteristic or range of characteristics which are exhibited by cells expressing physiologically normal levels of sphingosine kinase. In this regard, it should be understood that physiologically normal levels of sphingosine kinase will equate to a range of levels depending on whether a given endothelial cell is in a quiescent or activated state. Accordingly, the range of functional characteristics which an endothelial cell can perform will be usually defined by the state of differentiation of the endothelial cell and the level of expression of sphingosine kinase.

Without limiting the present invention to any one theory or mode of action, where physiologically normal levels of sphingosine kinase are expressed, a vascular endothelial cell may exhibit one or more characteristics including, but not limited to:
(i) the maintenance of a viable but quiescent state
(ii) the capacity to differentiate under appropriate stimulatory conditions (for example, maturation from CD34⁺ progenitor state to a more mature endothelial cell phenotype)
(iii) the capacity to proliferate
(iv) the maintenance of viability in an activated state
(v) the capacity to modulate cell surface molecule expression, such as adhesion molecule expression (for example, as an indicator of maturation or activation state)
(vi) the capacity to respond to cytokine stimulation
(vii) the capacity to bind neutrophils
(viii) the capacity to differentiate to a pro-inflammatory and/or angiogenic phenotype.

The present invention is directed to modulating these functional characteristics which can be observed under normal physiological conditions. It should be understood, however, that under normal physiological conditions there are certain inherent functional limitations to which endothelial cells are subject. For example, in order to maintain viability, vascular endothelial cells require exposure to certain anti-apoptotic signals such as those which are generated as a result of normal vascular endothelial cell attachment and cell spreading. Accordingly, in the absence of such signals—as may occur where cells are grown in vitro in suspension—unwanted apoptosis will occur. In another example, whereas immature, quiescent endothelial cells express the cell surface haematoprogenitor marker CD34, the stimulation and induction of endothelial cell proliferation (for example, in order to facilitate angiogenesis) results in loss of CD34 expression and, by definition, the development of an irreversible and more mature phenotype. In certain circumstances, such as where one is seeking to expand the CD34⁺ endothelial cell population, this can prove to be a disadvantage since the signals which initiate proliferation also lead to phenotypic maturation.

Accordingly, in a preferred embodiment, the subject functional characteristics are any one or more of the functional characteristics detailed in points (i) to (viii), above.

As detailed hereinbefore, it has also been determined that over-expressing sphingosine kinase in an endothelial cell can result in the induction of functional characteristics which are not generally observed when sphingosine kinase is expressed in the normal range. Accordingly, reference to “modulating” the functional characteristics of an endothelial cell “relative to” normal endothelial cell characteristics should be understood to mean that the over-expression of sphingosine kinase levels results in the induction of one or more characteristics which are not generally observed in the context of cells expressing sphingosine kinase in the normal range. It should be understood, however, that the subject characteristics may replace entirely the range of normal functional characteristics of an endothelial cell or one or more of these characteristics may be expressed together with one or more normal characteristics. Without limiting the present invention in any way, examples of characteristics which may be induced in endothelial cells over-expressing sphingosine kinase levels include, but are not limited to:
improved proliferative characteristics both in terms of an increased rate/extent of proliferation and the requirement for only minimal environmental/cell culture conditions under which proliferation can occur (herein referred to as “enhanced proliferation”)
improved cell viability. This may occur either at the level of down regulating apoptosis or preventing or otherwise induced cell death. For example, cell survival under conditions of stress (such as the removal of tissue culture supplements in the in vitro environment) is facilitated as is the down regulation of apoptosis which would normally occur in the absence of the anti-apoptotic signals which are provided as a result of integrin receptor engagement during matrix attachment and cell spreading. This is particularly relevant, for example, where in vitro cell culture populations are required to be maintained in suspension (herein referred to as “enhanced viability”).
changed differentiation pathways. In particular, whereas the CD34 haematoprogenitor cell surface marker is down regulated upon stimulation of endothelial cell progenitor proliferation or the proliferation of quiescent CD34⁺ endothelial cells, over-expression of sphingosine kinase results in maintenance of both CD34 expression and the progenitor phenotype of these cells despite the onset of proliferation/expansion (herein referred to as “maintaining the CD34⁺ endothelial cell progenitor phenotype”).

The subject functional characteristic modulation is therefore preferably:
(i) enhanced proliferation;
(ii) enhanced viability; and/or
(iii) maintaining the CD34⁺ endothelial cell progenitor phenotype.

Accordingly, the present invention also provides a method of modulating one or more endothelial cell functional characteristics, said method comprising modulating the functional level of sphingosine kinase wherein up-regulating said sphingosine kinase level modulates one or more of the functional characteristics of said endothelial cell relative to normal endothelial cell functional characteristics.

Preferably, said endothelial cell is a vascular endothelial cell.

In one preferred embodiment there is provided a method of modulating vascular endothelial cell proliferation, said method comprising modulating the functional level of sphingosine kinase wherein inducing over-expression of said sphingosine kinase level enhances the proliferation of said endothelial cell relative to normal endothelial cell proliferation.

In another embodiment there is provided a method of modulating vascular endothelial viability, said method comprising modulating the functional level of sphingosine kinase wherein inducing over-expression of said sphingosine kinase level enhances the viability of said vascular endothelial cell relative to normal endothelial cell viability.

In yet another preferred embodiment there is provided a method of modulating the CD34⁺ endothelial cell progenitor phenotype, said method comprising modulating the functional level of sphingosine kinase wherein inducing over-expression of said sphingosine kinase level maintains the CD34⁺ endothelial cell progenitor phenotype.

In accordance with these preferred embodiments, most preferably said modulation is up regulation of the subject functional characteristic.

Reference to “sphingosine kinase” should be understood as reference to all forms of this protein and to functional derivatives, homologues, analogues, chemical equivalents or mimetics thereof. This includes, for example, any isoforms which arise from alternative splicing of the subject sphingosine kinase mRNA or functional mutants or polymorphic variants of these proteins.

As detailed hereinbefore, it has been determined that inducing levels of intracellular sphingosine kinase which are higher than the basal levels which are observed in an unactivated or unstimulated endothelial cell results in the induction of unique functional characteristics. Accordingly, reference to “functional level” of sphingosine kinase should be understood as a reference to the level of sphingosine kinase activity which is present in any given cell as opposed to the concentration of sphingosine kinase, per se. Although an increase in the intracellular concentration of sphingosine kinase will generally correlate to an increase in the level of sphingosine kinase functional activity which is observed in a cell, the person skilled in the art would also understand that increases in the level of activity can be achieved by means other than merely increasing absolute intracellular sphingosine kinase concentrations. For example, one might utilise forms of sphingosine kinase which exhibit an increased half life or otherwise exhibit enhanced activity. Reference to “over-expressing” the subject sphingosine kinase level should therefore be understood as a reference to up regulating intracellular sphingosine kinase to an effective functional level which is greater than that expressed under the normal physiological conditions for a given endothelial cell or to the up-regulation of sphingosine kinase levels to any level of functionality but where that up-regulation event is one which is artificially effected rather than being an increase which has occurred in the subject cell due to the effects of naturally occurring physiology. Accordingly, this latter form of up-regulation may correlate to up-regulating sphingosine kinase to levels which fall within the normal physiological range but which are higher than pre-stimulation levels. The means by which up-regulation is achieved may be artificial means which seek to mimic a physiological pathway—for example introducing a hormone or other stimulatory molecule. Accordingly, the term “expressing” is not intended to be limited to the notion of sphingosine kinase gene transcription and translation. Rather, and as discussed in more detail hereinafter, it is a reference to an outcome, being the establishment of a higher and effective functional level of sphingosine kinase than is found under normal physiological conditions in an endothelial cell at a particular point in time (ie. as detailed hereinbefore, it includes non-naturally occurring increases in sphingosine kinase level, even where those increases may fall within the normal physiological range which one might observe). Reference to the subject functional level being an “effective” level should be understood as a level of over-expression which achieves the modulation of one or more functional characteristics of an endothelial cell relative to a normal endothelial cell. Without limiting the present invention to any one theory or mode of action, it has been determined that different levels of sphingosine kinase over-expression will induce specific and distinct cellular changes.

Reference to “modulating” in the context of endothelial cell functional characteristics should be understood as a reference to inducing the functional characteristics as detailed hereinbefore. In the context of the functional level of sphingosine kinase, reference to “modulating” should be understood as a reference to up regulating or down regulating the functional level of sphingosine kinase. Determining the specific optimum level (i.e. “effective” level) to which the sphingosine kinase should be up or down-regulated in order to achieve the desired phenotypic change for any given endothelial cell type is a matter of routine procedure. The person of skill in the art would be familiar with methods of determining such a level.

In one embodiment the present invention is directed to up regulating the functional level of sphingosine kinase as a means of introducing unique functional characteristics to a population of endothelial cells. However, it should nevertheless be understood that there are circumstances in which it is desirable to down regulate the ftuctional level of sphingosine kinase in order to obviate the expression of these characteristics. For example, one may seek to up regulate the functional level of sphingosine kinase in the context of a defined population of endothelial cells for a period of time sufficient to achieve a particular objective. However, once that objective has been achieved one would likely seek to down regulate the intracellular functional level of sphingosine kinase, to the extent that it is not transient, such that it is no longer over-expressed and the subject endothelial cells thereby take on a normal phenotype. In another example, one may identify certain disease conditions which are in fact characterised by an over-expression of the functional level of sphingosine kinase, for example due to the impact of genetic mutations. In such a situation, one may observe uncontrolled endothelial cell proliferation (angiogenesis) which could lead to tumour formation. Where such a situation exists, one may seek to down regulate the functional level of sphingosine kinase as a means of restoring a normal phenotypic profile to the endothelial cells in issue. In another example, down-regulation of sphingosine kinase levels in inflammatory conditions may be desirable where the subject inflammation is due to the occurrence of an endothelial cell inflammatory phenotype. In a particularly relevant example, rheumatoid arthritis is characterised by the development of both an angiogenic and an inflammatory endothelial cell phenotype. Accordingly, down-regulation of endothelial cell sphingosine kinase levels would be desirable as a therapeutic treatment. The present invention should therefore be understood to be directed to up regulating the sphingosine kinase functional level in order to introduce unique phenotypic properties to the population of endothelial cells and down-regulating a naturally or non-naturally induced state of sphingosine kinase over-expression.

As detailed above, reference to “modulating” sphingosine kinase functional levels is a reference to either up regulating or down regulating these levels. Such modulation may be achieved by any suitable means and includes:
(i) Modulating absolute levels of the active or inactive forms of sphingosine kinase (for example increasing or decreasing intracellular sphingosine kinase concentrations) such that either more or less sphingosine kinase is available for activation and/or to interact with its downstream targets.
(ii) Agonising or antagonising sphingosine kinase such that the flnctional effectiveness of any given sphingosine kinase molecule is either increased or decreased. For example, increasing the half life of sphingosine kinase may achieve an increase in the overall level of sphingosine kinase activity without actually necessitating an increase in the absolute intracellular concentration of sphingosine kinase. Similarly, the partial antagonism of sphingosine kinase, for example by coupling sphingosine kinase to a molecule that introduces some steric hindrance in relation to the binding of sphingosine kinase to its downstream targets, may act to reduce, although not necessarily eliminate, the effectiveness of sphingosine kinase signalling. Accordingly, this may provide a means of down-regulating sphingosine kinase functioning without necessarily down-regulating absolute concentrations of sphingosine kinase.

In terms of achieving the up or down-regulation of sphingosine kinase functioning, means for achieving this objective would be well known to the person of skill in the art and include, but are not limited to:
(i) Introducing into a cell a nucleic acid molecule encoding sphingosine kinase or functional equivalent, derivative or analogue thereof in order to up-regulate the capacity of said cell to express sphingosine kinase.
(ii) Introducing into a cell a proteinaceous or non-proteinaceous molecule which modulates transcriptional and/or translational regulation of a gene, wherein this gene may be a sphingosine kinase gene or functional portion thereof or some other gene which directly or indirectly modulates the expression of the sphingosine kinase gene.
(iii) Introducing into a cell the sphingosine kinase expression product (in either active or inactive form) or a functional derivative, homologue, analogue, equivalent or mimetic thereof.
(iv) Introducing a proteinaceous or non-proteinaceous molecule which functions as an antagonist to the sphingosine kinase expression product.
(v) Introducing a proteinaceous or non-proteinaceous molecule which functions as an agonist of the sphingosine kinase expression product.

The proteinaceous molecules described above may be derived from any suitable source such as natural, recombinant or synthetic sources and includes fusion proteins or molecules which have been identified following, for example, natural product screening. The reference to non-proteinaceous molecules may be, for example, a reference to a nucleic acid molecule or it may be a molecule derived from natural sources, such as for example natural product screening, or may be a chemically synthesised molecule. The present invention contemplates analogues of the sphingosine kinase expression product or small molecules capable of acting as agonists or antagonists. Chemical agonists may not necessarily be derived from the sphingosine kinase expression product but may share certain conformational similarities. Alternatively, chemical agonists may be specifically designed to meet certain physiochemical properties. Antagonists may be any compound capable of blocking, inhibiting or otherwise preventing sphingosine kinase from carrying out its normal biological function, such as molecules which prevent its activation or else prevent the downstream functioning of activated sphingosine kinase. Antagonists include monoclonal antibodies and antisense nucleic acids which prevent transcription or translation of sphingosine kinase genes or mRNA in mammalian cells. Modulation of expression may also be achieved utilising antigens, RNA, ribosomes, DNAzymes, RNA aptamers, antibodies or molecules suitable for use in cosuppression. The proteinaceous and non-proteinaceous molecules referred to in points (i)-(v), above, are herein collectively referred to as “modulatory agents”.

Screening for the modulatory agents hereinbefore defined can be achieved by any one of several suitable methods including, but in no way limited to, contacting a cell comprising the sphingosine kinase gene or functional equivalent or derivative thereof with an agent and screening for the modulation of sphingosine kinase protein production or functional activity, modulation of the expression of a nucleic acid molecule encoding sphingosine kinase or modulation of the activity or expression of a downstream sphingosine kinase cellular target. Detecting such modulation can be achieved utilising techniques such as Western blotting, electrophoretic mobility shift assays and/or the readout of reporters of sphingosine kinase activity such as luciferases, CAT and the like.

It should be understood that the sphingosine kinase gene or functional equivalent or derivative thereof may be naturally occurring in the cell which is the subject of testing or it may have been transfected into a host cell for the purpose of testing. Further, the naturally occurring or transfected gene may be constitutively expressed—thereby providing a model useful for, inter alia, screening for agents which down regulate sphingosine kinase activity, at either the nucleic acid or expression product levels, or the gene may require activation—thereby providing a model useful for, inter alia, screening for agents which up regulate sphingosine kinase expression. Further, to the extent that a sphingosine kinase nucleic acid molecule is transfected into a cell, that molecule may comprise the entire sphingosine kinase gene or it may merely comprise a portion of the gene such as the portion which regulates expression of the sphingosine kinase product. For example, the sphingosine kinase promoter region may be transfected into the cell which is the subject of testing. In this regard, where only the promoter is utilised, detecting modulation of the activity of the promoter can be achieved, for example, by ligating the promoter to a reporter gene. For example, the promoter may be ligated to luciferase or a CAT reporter, the modulation of expression of which gene can be detected via modulation of fluorescence intensity or CAT reporter activity, respectively.

In another example, the subject of detection could be a downstream sphingosine kinase regulatory target, rather than sphingosine kinase itself. Yet another example includes sphingosine kinase binding sites ligated to a minimal reporter. For example, modulation of sphingosine kinase activity can be detected by screening for the modulation of the functional activity in an endothelial cell. This is an example of an indirect system where modulation of sphingosine kinase expression, per se, is not the subject of detection. Rather, modulation of the molecules which sphingosine kinase regulates the expression of, are monitored.

These methods provide a mechanism for performing high throughput screening of putative modulatory agents such as the proteinaceous or non-proteinaceous agents comprising synthetic, combinatorial, chemical and natural libraries. These methods will also facilitate the detection of agents which bind either the sphingosine kinase nucleic acid molecule or expression product itself or which modulate the expression of an upstream molecule, which upstream molecule subsequently modulates sphingosine kinase expression or expression product activity. Accordingly, these methods provide a mechanism for detecting agents which either directly or indirectly modulate sphingosine kinase expression and/or activity.

The agents which are utilised in accordance with the method of the present invention may take any suitable form. For example, proteinaceous agents may be glycosylated or unglycosylated, phosphorylated or dephosphorylated to various degrees and/or may contain a range of other molecules used, linked, bound or otherwise associated with the proteins such as amino acids, lipid, carbohydrates or other peptides, polypeptides or proteins. Similarly, the subject non-proteinaceous molecules may also take any suitable form. Both the proteinaceous and non-proteinaceous agents herein described may be linked, bound otherwise associated with any other proteinaceous or non-proteinaceous molecules. For example, in one embodiment of the present invention, said agent is associated with a molecule which permits its targeting to a localised region.

The subject proteinaceous or non-proteinaceous molecule may act either directly or indirectly to modulate the expression of sphingosine kinase or the activity of the sphingosine kinase expression product. Said molecule acts directly if it associates with the sphingosine kinase nucleic acid molecule or expression product to modulate expression or activity, respectively. Said molecule acts indirectly if it associates with a molecule other than the sphingosine kinase nucleic acid molecule or expression product which other molecule either directly or indirectly modulates the expression or activity of the sphingosine kinase nucleic acid molecule or expression product, respectively. Accordingly, the method of the present invention encompasses the regulation of sphingosine kinase nucleic acid molecule expression or expression product activity via the induction of a cascade of regulatory steps.

The term “expression” in this context refers to the transcription and translation of a nucleic acid molecule. Reference to “expression product” is a reference to the product produced from the transcription and translation of a nucleic acid molecule.

“Derivatives” of the molecules herein described (for example sphingosine kinase or other proteinaceous or non-proteinaceous agents) include fragments, parts, portions or variants from either natural or non-natural sources. Non-natural sources include, for example, recombinant or synthetic sources. By “recombinant sources” is meant that the cellular source from which the subject molecule is harvested has been genetically altered. This may occur, for example, in order to increase or otherwise enhance the rate and volume of production by that particular cellular source. Parts or fragments include, for example, active regions of the molecule. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in a sequence has been removed and a different residue inserted in its place. Additions to amino acid sequences include fusions with other peptides, polypeptides or proteins, as detailed above.

Derivatives also include fragments having particular epitopes or parts of the entire protein fused to peptides, polypeptides or other proteinaceous or non-proteinaceous molecules. For example, sphingosine kinase or derivative thereof may be fused to a molecule to facilitate its entry into a cell. Analogues of the molecules contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences which may be utilised in accordance with the method of the present invention may similarly be derived from single or multiple nucleotide substitutions, deletions and/or additions including fusion with other nucleic acid molecules. The derivatives of the nucleic acid molecules utilised in the present invention include oligonucleotides, PCR primers, antisense molecules, molecules suitable for use in cosuppression and fusion of nucleic acid molecules. Derivatives of nucleic acid sequences also include degenerate variants.

A “variant” of sphingosine kinase should be understood to mean molecules which exhibit at least some of the functional activity of the form of sphingosine kinase of which it is a variant. A variation may take any form and may be naturally or non-naturally occurring. A mutant molecule is one which exhibits modified functional activity.

A “homologue” is meant that the molecule is derived from a species other than that which is being treated in accordance with the method of the present invention. This may occur, for example, where it is determined that a species other than that which is being treated produces a form of sphingosine kinase which exhibits similar and suitable functional characteristics to that of the sphingosine kinase which is naturally produced by the subject undergoing treatment.

Chemical and functional equivalents should be understood as molecules exhibiting any one or more of the functional activities of the subject molecule, which functional equivalents may be derived from any source such as being chemically synthesised or identified via screening processes such as natural product screening. For example chemical or functional equivalents can be designed and/or identified utilising well known methods such as combinatorial chemistry or high throughput screening of recombinant libraries or following natural product screening.

For example, libraries containing small organic molecules may be screened, wherein organic molecules having a large number of specific parent group substitutions are used. A general synthetic scheme may follow published methods (eg., Bunin B A, et al. (1994) Proc. Natl. Acad. Sci. USA, 91:4708-4712; DeWitt S H, et al. (1993) Proc. Natl. Acad. Sci. USA, 90:6909-6913). Briefly, at each successive synthetic step, one of a plurality of different selected substituents is added to each of a selected subset of tubes in an array, with the selection of tube subsets being such as to generate all possible permutation of the different substituents employed in producing the library. One suitable permutation strategy is outlined in U.S. Pat. No. 5,763,263.

There is currently widespread interest in using combinational libraries of random organic molecules to search for biologically active compounds (see for example U.S. Pat. No. 5,763,263). Ligands discovered by screening libraries of this type may be useful in mimicking or blocking natural ligands or interfering with the naturally occurring ligands of a biological target. In the present context, for example, they may be used as a starting point for developing sphingosine kinase analogues which exhibit properties such as more potent pharmacological effects. Sphingosine kinase or a fumctional part thereof may according to the present invention be used in combination libraries formed by various solid-phase or solution-phase synthetic methods (see for example U.S. Pat. No. 5,763,263 and references cited therein). By use of techniques, such as that disclosed in U.S. Pat. No. 5,753,187, millions of new chemical and/or biological compounds may be routinely screened in less than a few weeks. Of the large number of compounds identified, only those exhibiting appropriate biological activity are further analysed.

With respect to high throughput library screening methods, oligomeric or small-molecule library compounds capable of interacting specifically with a selected biological agent, such as a biomolecule, a macromolecule complex, or cell, are screened utilising a combinational library device which is easily chosen by the person of skill in the art from the range of well-known methods, such as those described above. In such a method, each member of the library is screened for its ability to interact specifically with the selected agent. In practising the method, a biological agent is drawn into compound-containing tubes and allowed to interact with the individual library compound in each tube. The interaction is designed to produce a detectable signal that can be used to monitor the presence of the desired interaction. Preferably, the biological agent is present in an aqueous solution and further conditions are adapted depending on the desired interaction. Detection may be performed for example by any well-known functional or non-functional based method for the detection of substances.

In addition to screening for molecules which mimic the activity of sphingosine kinase, it may also be desirable to identify and utilise molecules which function agonistically or antagonistically to sphingosine kinase in order to up or down-regulate the functional activity of sphingosine kinase in relation to modulating endothelial cell growth. The use of such molecules is described in more detail below. To the extent that the subject molecule is proteinaceous, it may be derived, for example, from natural or recombinant sources including fusion proteins or following, for example, the screening methods described above. The non-proteinaceous molecule may be, for example, a chemical or synthetic molecule which has also been identified or generated in accordance with the methodology identified above. Accordingly, the present invention contemplates the use of chemical analogues of sphingosine kinase capable of acting as agonists or antagonists. Chemical agonists may not necessarily be derived from sphingosine kinase but may share certain conformational similarities. Alternatively, chemical agonists may be specifically designed to mimic certain physiochemical properties of sphingosine kinase. Antagonists may be any compound capable of blocking, inhibiting or otherwise preventing sphingosine kinase from carrying out its normal biological functions. Antagonists include monoclonal antibodies specific for sphingosine kinase or parts of sphingosine kinase.

Analogues of sphingosine kinase or of sphingosine kinase agonistic or antagonistic agents contemplated herein include, but are not limited to, modifications to side chains, incorporating unnatural amino acids and/or derivatives during peptide, polypeptide or protein synthesis and the use of crosslinkers and other methods which impose conformational constraints on the analogues. The specific form which such modifications can take will depend on whether the subject molecule is proteinaceous or non-proteinaceous. The nature and/or suitability of a particular modification can be routinely determined by the person of skill in the art.

For example, examples of side chain modifications contemplated by the present invention include modifications of amino groups such as by reductive alkylation by reaction with an aldehyde followed by reduction with NaBH4; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide. Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl- -aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nehep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(ρ-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH₂)n spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The method of the present invention contemplates the modulation of endothelial cell functioning both in vitro and in vivo. Although the preferred method is to treat an individual in vivo, it should nevertheless be understood that it may be desirable that the method of the invention be applied in an in vitro environment. For example, one may seek to initiate angiogenesis by inducing endothelial cell proliferation in accordance with the method of the present invention in a donor graft prior to its introduction to a host. In another example, one may seek to expand populations of endothelial cells in culture prior to their localised introduction to a subject who is undergoing treatment. In yet another example, the method of the present invention may be utilised to create cell lines.

Accordingly, another aspect of the present invention is directed to a method of modulating one or more endothelial cell functional characteristics in a mammal, said method comprising modulating the functional level of sphingosine kinase wherein inducing over-expression of said sphingosine kinase level modulates one or more of the functional characteristics of said endothelial cell.

More particularly, said method is directed to modulating one or more vascular endothelial cell functional characteristics in a mammal, said method comprising modulating the functional level of sphingosine kinase in said mammal wherein inducing over-expression of said sphingosine kinase level modulates one or more of the functional characteristics of said endothelial cell.

Still more particularly, said vascular endothelial cell is a CD34⁺ endothelial cell.

Preferably, said functional characteristics are one or more of:
(i) the maintenance of a viable but quiescent state
(ii) the capacity to differentiate under appropriate stimulatory conditions (for example, maturation from CD34⁺ progenitor state to a more mature endothelial cell phenotype)
(iii) the capacity to proliferate
(iv) the maintenance of viability in an activated state
(v) the capacity to modulate cell surface molecule expression, such as adhesion molecule expression (for example, as an indicator of maturation or activation state)
(vi) the capacity to respond to cytokine stimulation
(vii) the capacity to bind neutrophils
(viii) the capacity to differentiate to a pro-inflammatory and/or angiogenic phenotype.

The present invention also provides a method of modulating one or more endothelial cell functional characteristics, said method comprising modulating the functional level of sphingosine kinase wherein up-regulating said sphingosine kinase level modulates one or more of the functional characteristics of said endothelial cell relative to normal endothelial cell functional characteristics.

In one preferred embodiment, there is provide a method of modulating vascular endothelial cell proliferation in a mammal, said method comprising modulating the functional level of sphingosine kinase in said mammal wherein inducing over-expression of said sphingosine kinase level enhances the proliferation of said endothelial cell relative to normal endothelial cell proliferation.

In another preferred embodiment, there is provided the method of modulating vascular endothelial cell viability in a mammal, said method comprising modulating the functional level of sphingosine kinase in said manmmal wherein inducing over-expression of said sphingosine kinase level enhances the viability of said vascular endothelial cell relative to normal endothelial cell viability.

In yet another preferred embodiment, there is provided a method of modulating the CD34⁺ endothelial cell progenitor phenotype in a mammal, said method comprising modulating the functional level of said sphingosine kinase in said mammal wherein inducing over-expression of said sphingosine kinase level maintains the CD34⁺ endothelial cell progenitor phenotype.

A further aspect of the present invention relates to the use of the invention in relation to the treatment and/or prophylaxis of disease conditions or other unwanted conditions. Without limiting the present invention to any one theory or mode of action, the development of methodology which facilitates enhancement of endothelial cell proliferation, viability and the maintenance of the progenitor CD34⁺ endothelial cell phenotype and the modulation of the endothelial cell inflammatory and angiogenic phenotypes provides a means of rapidly and efficiently expanding endothelial cell populations either in vitro or in vivo. For example, the fact that the viability of these cells can be enhanced renders the invention particularly useful in situations where ideal environmental factors may not be present. In this regard, the inventors have developed herewith a means of generating particularly robust populations of endothelial cells. In particularly preferred embodiments, the method of the present invention may be utilised to establish vascular grafts, to induce or seed vascularisation of tissue or organ grafts or to induce vascularisation of de-vascularised regions such as regions of amyloid plaque deposition. In another example the method of the present invention could be utilised to deliver drugs to the vascular system via endothelial cells which may require the phenotypic features induced by sphingosine kinase over-expression in order to provide the desired survival or maturation conditions. Further, maintaining populations of immature endothelial cells may be useful to the extent that such cells are required in order to facilitate their stimulation and differentiation along a particular cell lineage, even a non-vascular cell lineage such as the differentiation to muscle cells. Sphingosine kinase over-expression would be useful in this context since populations of immature proliferating endothelial cells could be maintained in a effective manner. Still further, down-regulation of the inflammatory and/or angiogenic phenotype in inflammatory conditions such as rheumatoid arthritis would be desirable.

The present invention therefore contemplates a method for the treatment and/or prophylaxis of a condition characterised by aberrant or otherwise unwanted endothelial cell functioning in a mammal, said method comprising modulating the functional level of sphingosine kinase in said mammal wherein inducing over-expression of said sphingosine kinase level up-regulates one or more functional characteristics of said endothelial cells.

Reference to “aberrant or otherwise unwanted endothelial cell functioning” should be understood as a reference to under active endothelial cell functioning, overactive endothelial cell functioning, to physiologically normal functioning which is inappropriate in that it is too low or to the absence of functioning. In this regard, reference to “functioning” should be understood as a reference to any one or more of the normal functional characteristics as hereinbefore defined. Reference to “inadequate functioning” should also be understood to include reference to the presence of insufficient numbers of progenitor cells to differentiate along the endothelial cell pathway. For example, in certain situations, such as wound healing and tissue/organ transplantation, there may be very low levels of CD34⁺ progenitor cells available to differentiate along the endothelial cell pathway. The method of the present invention provides a means of not only generating endothelial cell progenitor expansion, but also means of maintaining a population of these progenitor cells, despite the onset of proliferation.

More particularly, the present invention provides the method for the treatment and/or prophylaxis of a condition characterised by aberrant or otherwise unwanted vascular endothelial cell functioning in a mammal, said method comprising modulating the fuinctional level of sphingosine kinase in said mammal wherein inducing over-expression of said sphingosine kinase level up-regulates one or more functional characteristics of said endothelial cells.

Preferably said condition is vascular engraftment, wound repair, tissue/organ transplantation or the repair of devascularised tissue and said sphingosine kinase modulating is up-regulation. In a most preferred embodiment, said up-regulated functional characteristic is one or more of enhanced endothelial cell proliferation, enhanced endothelial cell viability and/or maintenance of the CD34⁺ endothelial cell progenitor phenotype.

In another preferred embodiment, said condition is an inflammatory condition and said sphingosine kinase modulation is down-regulation. Most preferably, said down-regulated functional characteristic is down-regulation of en endothelial cell inflammatory and/or angiogenic phenotype.

In yet another preferred embodiment, said condition is characterised by unwanted angiogenesis and said sphingosine kinase modulation is down-regulation. Most preferably said down-regulated functional characteristic is endothelial cell angiogenic phenotype and said condition is a tumour.

In a most preferred embodiment, there is provided the method for the treatment and/or prophylaxis of a condition characterised by aberrant or otherwise unwanted vascular endothelial cell functioning in a mammal, said method comprising administering to said mammal an effective amount of an agent for a time and under conditions sufficient to modulate the functional level of sphingosine kinase.

Reference to “agent” should be understood to have the same meaning as hereinbefore defined. However, in the context of this aspect of the present invention reference to “agent” should also be understood as a reference to a population of endothelial cells which have been treated in accordance with the method of the present invention. For example, prophylactically or therapeutically treating a condition characterised by inadequate vascular endothelial cell functioning may be achieved by introducing to the patient a population of endothelial cells which exhibit one or more of the improved functional characteristics which are obtainable in accordance with the method of the present invention. For example, a population of suitably treated CD34⁺ endothelial cell progenitors may be introduced to a site which requires revascularisation such as a site of wound repair or a site of abnormal devascularisation (such as would occur where arnyloid plaques are deposited).

An “effective amount” means an amount necessary at least partly to attain the desired response, or to delay the onset or inhibit progression or halt altogether, the onset or progression of the particular condition being treated. The amount varies depending upon the health and physical condition of the individual to be treated, the taxonomic group of the individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity or onset of a particular condition. “Treatment” may also reduce the severity of an existing condition.

The present invention further contemplates a combination of therapies, such as the administration of the modulatory agent together with other proteinaceous or non-proteinaceous molecules which may facilitate the desired therapeutic or prophylactic outcome.

Administration of molecules of the present invention hereinbefore described [herein collectively referred to as “modulatory agent”], in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (e.g. using slow release molecules). The modulatory agent may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant. Preferably, said route of administration is oral.

In accordance with these methods, the agent defined in accordance with the present invention may be coadministered with one or more other compounds or molecules. By “coadministered” is meant simultaneous administration in the same forrnulation or in two different formulations via the same or different routes or sequential administration by the same or different routes. For example, the subject sphingosine kinase may be administered together with an agonistic agent in order to enhance its effects. Alternatively, in the case of organ tissue transplantation, the sphingosine kinase may be administered together with immunosuppressive drugs. By “sequential” administration is meant a time difference of from seconds, minutes, hours or days between the administration of the two types of molecules. These molecules may be administered in any order.

Another aspect of the present invention relates to the use of an agent capable of modulating the functional level of sphingosine kinase in the manufacture of a medicament for the modulation of one or more endothelial cell functional characteristics in a mammal wherein inducing over-expression of said sphingosine kinase level modulates one or more of the functional characteristics of said endothelial cells.

In another aspect, the present invention relates to the use of sphingosine kinase or a nucleic acid encoding sphingosine kinase in the manufacture of a medicament for the modulation of one or more endothelial cell functional characteristics in a mammal wherein inducing over-expression of said sphingosine kinase level modulates one or more of the functional characteristics of said endothelial cells.

According to these preferred embodiments, the subject endothelial cells are preferably vascular endothelial cells and even more preferably, CD34⁺ vascular endothelial cells.

Even more preferably, said medicament is used to treat a condition characterised by aberrant or unwanted endothelial cell functioning as hereinbefore described.

The term “mammal” and “subject” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal Even more preferably, the mammal is a human.

In yet another further aspect, the present invention contemplates a pharmaceutical composition comprising the modulatory agent as hereinbefore defined and one or more pharmaceutically acceptable carriers and/or diluents. Said agents are referred to as the active ingredients

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

The pharmaceutical composition may also comprise genetic molecules such as a vector capable of transfecting target cells where the vector carries a nucleic acid molecule encoding sphingosine kinase or a modulatory agent as hereinbefore defined. The vector may, for example, be a viral vector. The pharmaceutical composition may also comprise endothelial cell populations which have been treated in accordance with the method of the present invention.

Still another aspect of the present invention is directed to a method of generating an endothelial cell, which endothelial cell is characterised by the modulation of one or more functional characteristics relative to normal endothelial cell functional characteristics, said method comprising inducing over-expression of the functional level of sphingosine kinase in said cell.

Yet another aspect of the present invention is directed to the endothelial cells which are generated in accordance with the methods defined herein.

Still yet another aspect of the present invention is directed to the use of endothelial cells developed in accordance with the method defined herein in the treatment and/or prophylaxis of conditions characterised by inadequate endothelial cell functioning.

Further features of the present invention are more fully described in the following non-limiting figures and examples.

### EXAMPLE 1

### Raised Intracellular Levels of Sphingosine Kinase Enhance Cell Survival Thorugh Targeted Regulation of PECAM-1

### Material and Methods

Transfection of HUVEC

HUVEC were isolated and cultured as previously described (Litwin M, Clark K, Noack L, Furze J, Berndt M, Albelda S et al. (1997) J Cell Biol 139(1):219-228), with medium supplemented with 50 g/ml endothelial growth supplement (Collaborative Research, MA, USA) and 50 g/ml heparin (Sigma, St Louis, Mo., USA).

Adenovirus Production and Generation of HUVEC Over-expressing SK

The AdEasy system was used to produce recombinant adenovirus carrying SK (or empty vector, EV) according to the Qbiogene Version 1.4 AdEasy™ Vector system manual (http:www.qbiogene.com/products/adenovirus/adeasy.shtml). 293 cells were cultured in 25 cm² flasks in complete Dulbecco's modified Eagle's medium (CSL Biosciences, Parkville, Australia) containing 10% fetal calf serum (FCS). Virus was amplified in 293 cells and purified on a cesium chloride gradient with centrifugation. The viral titre was determined using the TCID₅₀ method according to the manufacturer's protocol. Transient transfection of HUVEC was achieved by infection with adenoviral preparations of SK or EV using equivalent plaque forming units (pfu)/cell which yielded a similar level of GFP expression.

Cells were used for functional assays 24-72 hours post-transfection. Over-expression of SK was confirmed with both Western blot, and SK activity assay.

Western Blotting

SDS-polyacrylamide gel electrophoresis was performed as described (Pitson S M, Moretti P A, Zebol J R, Xia P, Gamble J R, Vadas M A et al. (2000) J Biol Chem; 275(43):33945-33950) on cell lysates using 12% acrylamide gels. Proteins were transferred to PVDF membranes, blocked in 5% low fat milk in PBS with 0.1% Tween20 for one hour, and incubated overnight at 4 C with M2 mouse anti-FLAG antibody (Sigma, St Louis, Mo.), rabbit polyclonal anti-phospho-Akt (Cell Signaling Technology), rabbit polyclonal anti-Akt (Cell Signaling Technology), anti-phosphotyrosine (Cell Signaling Technology), or for one hour at room temperature with mouse anti-cyclin D1 or cyclin E (Santa Cruz Biotechnology) or mouse monoclonal antibody directed to PECAM-1 (51-6F6) raised at The Hanson Institute, Adelaide, Australia. The membrane was incubated with horseradish peroxidase-conjugated anti-mouse IgG or anti-rabbit IgG (Pierce) and immunocomplexes were detected using enhanced chemiluminescence (Amersham Pharmacia Biotech).

SK Activity

SK activity was determined as previously described (Xia P, Gamble J R, Rye K A, Wang L, Hii C S, Cockerill P et al. (1998) Proc Natl Acad Sci USA; 95(24):14196-14201). Briefly, D-erythrosphingosine and [-³²P]ATP were used as substrates and were incubated with whole cell lysates. The labeled lipids were extracted and resolved by TLC. The radioactive spots were quantified by the Phosphoimage system.

Fluorescence Activated Cell Sorting (FACS)

Flow cytometric analysis of cell surface expression of PECAM-1 and VE-Cadherin was performed as previously described (Xia P, Gamble J R, Rye K A, Wang L, Hii C S, Cockerill P et al. (1998) supra) using 10 g/ml mouse monoclonal primary antibodies to PECAM-1 (51-6F6) or VE-Cadherin (55-7H1) generated in our laboratory (Gamble J R, Khew-Goodall Y, Vadas M A. (1993) J Immunol; 150(10):4494-4503). The secondary antibody used was goat anti-mouse IgG R-phycoerythrin conjugate, (Southern Biotech Birmingham, Ala., USA). The median fluorescence intensity was determined using a Coulter Epics Profile XL flow cytometer. FAC S analysis of the cell surface expression of CD34 was done by incubating 1×10⁶ cells with 10 L of anti-CD34, R-phycoerythrin (R-PE)-conjugated mouse anti-human mAb (BD Pharmingen, San Diego, Calif.) for 30 minutes at room temperature, and then determining the median fluorescence intensity.

Measurement of Caspase-3 Activity

Cell lysates were prepared as described (Laemmli U K. (1970) Nature; 227(259):680-685), using caspase-3 lysis buffer (10% NP-40, 1M Tris-HCL, 1M EDTA). Ten L of lysate was placed onto a 96 well tray. Ten mL of caspase-3 buffer (12 g/L Hepes, 100 g/L sucrose, 1 g/L Chaps, pH 7.4) was mixed with 15.45 mg DL-Dithiothreitol, (Sigma, St Louis, USA) and 10 L of 2.5 mM DEVD-AFC substrate (Calbiochem-Novabiochem, Darmstadt, Germany). This mixture (200 L) was added to each well and incubated for five hours. Fluorescence was measured with a well plate reader (excitation and emission wavelengths of 385 nm and 460 nm) and normalized for the protein concentration.

Immunofluorescent Staining of Apoptotic Cells

Cells were seeded into fibronectin coated LabTek slides at 6×10⁴ cells per well in medium comprising varying concentrations of FCS and incubated at 37 C for 24 hours. The cells were incubated at 37 C with 150 L DAPI-Methanol (Roche, Manheim, Germany) for 15 minutes and then washed with methanol. Apoptotic cells were visualized by immunofluorescent microscopy to stain very brightly, with fragmented nuclei, while live cells had intact nuclei and less intense staining. The percentage of apoptotic cells in consecutive fields was calculated.

Cell Permeability

Endothelial cells were seeded into fibronectin-coated 3.0 m transwells at 10×10⁴ cells per well, with 600 L culture medium added to the bottom of the transwell. FITCdextran (500 g/mL) was added to each transwell and then 20 L medium collected from the bottom of each transwell at predetermined time points and dispensed into a 96 well microtitre tray containing 60 L serum free medium per well. The fluorescence was determined using a well plate reader, using excitation and emission wavelengths of 485 nm and 530 nm.

Cell Survival

Endothelial cells were plated into gelatin coated 96 well microtitre trays at 3×10³ cells per well in serum-free medium. MTS (Promega, WI, USA) was used to measure cell viability. Optical density at 490 nm was measured on Day 0, Day 1, Day 2, and Day 3.

Cell Suspension

Cells were plated as above, in non-tissue culture, non-adhesive 96 well microtitre trays coated with 1% bovine serum albumin at 8×10³ cells per well, in serum free medium. The optical density was determined as above using MTS at Day 0, Day 1, Day 2, and Day 3.

Results

Over-expression of SK Enhanced SK Activity

To determine the effect on endothelial cell function of over-expression of SK, HUVEC were infected with adenovirus containing SK at 1 pfu/cell. Infection of HUVEC with 1 pfti/cell resulted in 5.17 (95% CI 4.86-5.51)-fold increase in SK activity above control which was statistically significant (p 0.001).

Over-expression of Sphingosine Kinase Enhances Cell Survival and Survival in Suspension

Cell survival was measured in serum-free medium supplemented with ECGs and in non-tissue culture non-adhesive trays coated with 1% bovine serum albumin under serum free culture conditions.

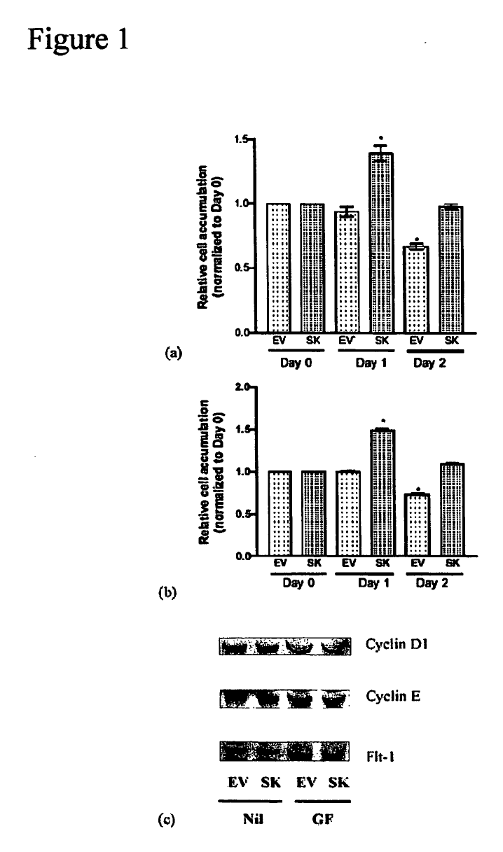

Cells over-expressing SK showed enhanced survival in serum free conditions (FIG. 1a) and when grown in suspension (FIG. 1b), compared with control cells. Twenty-four hours after plating, the cells over-expressing SK had increased in number. Even 48 hours after plating, more cells over-expressing SK survived either under SF conditions or in non-adherent conditions, compared with control cells. In contrast, cell numbers in EV cells were maintained for 24 hours, but rapidly dropped off thereafter. Cells over-expressing SK were visualized by microscopy to form aggregates in suspension, which were more extensive than those formed by control cells. Measurement of cyclins E and D showed no change in levels between cells over-expressing SK compared with EV cells (FIG. 1c), thus suggesting that the alteration in number seen in the cells over-expressing SK may be due to an anti-apoptotic effect.

Over-expression of SK Confers Resistance to Serum Deprivation-induced Apoptosis

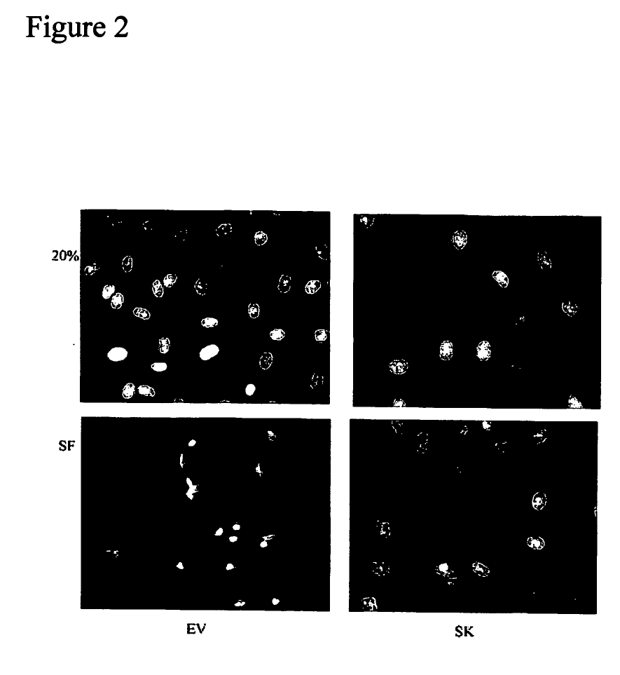

The resistance to serum deprivation induced apoptosis in cells over-expressing SK was confirmed by performing a DAPI stain under basal conditions and after 24 hours of serum deprivation. FIG. 2 shows that under basal conditions there was no difference in the number of apoptotic cells between cells over-expressing SK and control. With serum deprivation, control cells responded with a large increase in the number of apoptotic cells, while among cells over-expressing SK, there were negligible numbers of apoptotic cells

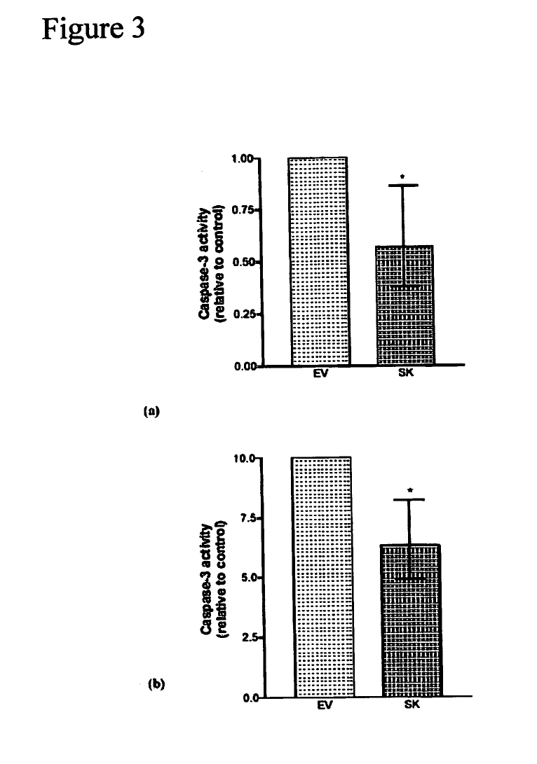

The results of DAPI staining were confirmed by measurement of caspase-3 activity under basal conditions and in response to 24 hours of serum deprivation. Over-expression of SK was shown to significantly reduce basal caspase-3 activity (FIG. 3a) and to confer further resistance to caspase-3 activation induced by serum deprivation (FIG. 3b).

Over-expression of SK Activates the PI-3K/Akt Pathway

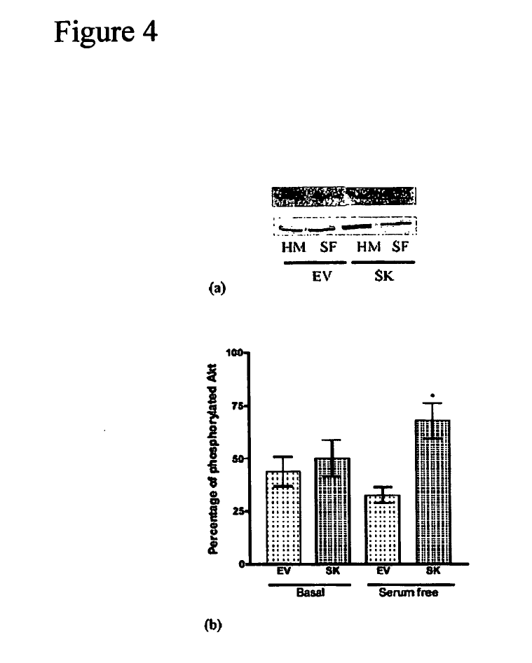

Survival factors such as growth factor and attachment to extracellular matrix influence cell survival through a number of pathways which include the PI-3K/Akt pathway. To determine whether this pathway is involved in the increased survival induced by over-expression of SK, phosphorylation of Akt was assessed. Under basal conditions there was no significant difference in the percentage of phosphorylated AKT (p-Akt) in cells over-expressing SK compared with control (p=0.47), as shown in FIG. 4(a). A reduction in the phosphorylation of Akt in response to serum deprivation was seen in EV cells. Cells over-expressing SK however responded to the stress of serum deprivation by a further increase in phosphorylation of Akt. Thus, in serum free conditions cells over-expressing SK had significantly greater phosphorylation of Akt than control, suggesting the activation of this pathway. This was confirmed and quantitated by ImageQuant software in five separate endothelial cell lines (FIG. 4(b)).

The PI-3 Kinase Pathway Mediates SK-induced Cell Survival

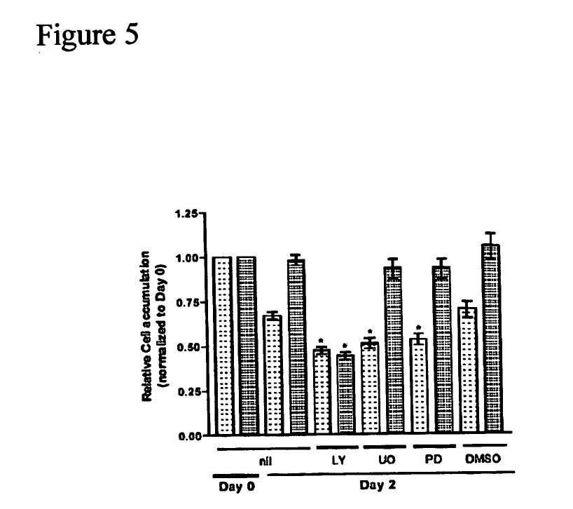

PI-3K is a known upstream regulator of Akt activation, and thus the effect of inhibiting PI-3K (with LY294002) on SK-mediated cell survival was investigated. SK-induced cell survival was abolished in the presence of LY294002 but not in the presence of either of two inhibitors of the MAPK pathway, U0126 or PD98059 (FIG. 5). Whilst LY294002, U0126 and PD98059 all significantly reduced cell survival of control cells, cells over-expressing SK responded to LY294002 with reduced cell survival but not to UO126 or PD98059. This indicates that SK-induced cell survival is mediated through the PI-3K pathway and that the MAPK pathway is not implicated. This is in contrast to S1P-mediated cell survival which involves the MAPK and PI-3K/Akt pathways.

Sphingosine Kinase Induces PECAM-1 Expression and Dephosphorylation

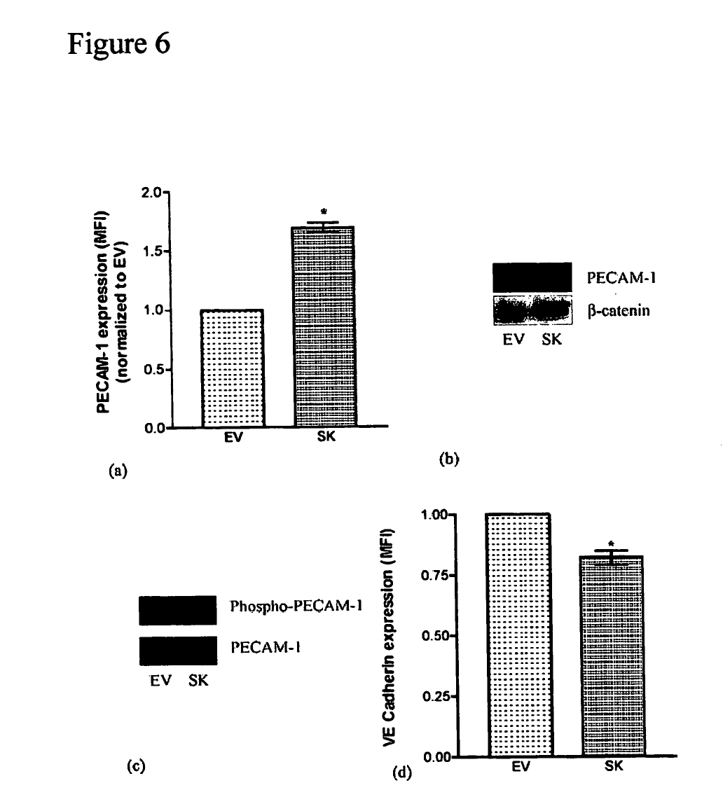

Over-expression of SK significantly increased cell surface expression of PECAM-1 compared with control as measured by flow cytometry (FIG. 6a). This was confirmed by Westem blot (FIG. 6b). Stimulation of normal HUVEC with exogenous S1P did not induce PECAM-1 expression. There was however no change in the other junctional protein, catenin (FIG. 6b) and a small reduction in VE cadherin (FIG. 6d).

In endothelial cells PECAM-1 is phosphorylated on tyrosine residues, and phosphorylation is one method of regulation of PECAM-1. Hence phosphorylation of PECAM-1 was measured by Western blot. Enforced expression of sphingosine kinase significantly reduced phosphorylation of PECAM-1 (FIG. 6c). In three separate endothelial cell lines, the mean fold percentage reduction in the proportion of PECAM-1 which was phosphorylated for cells over-expressing SK compared with control was 48% (95% CI 28-63%), p=0.054.

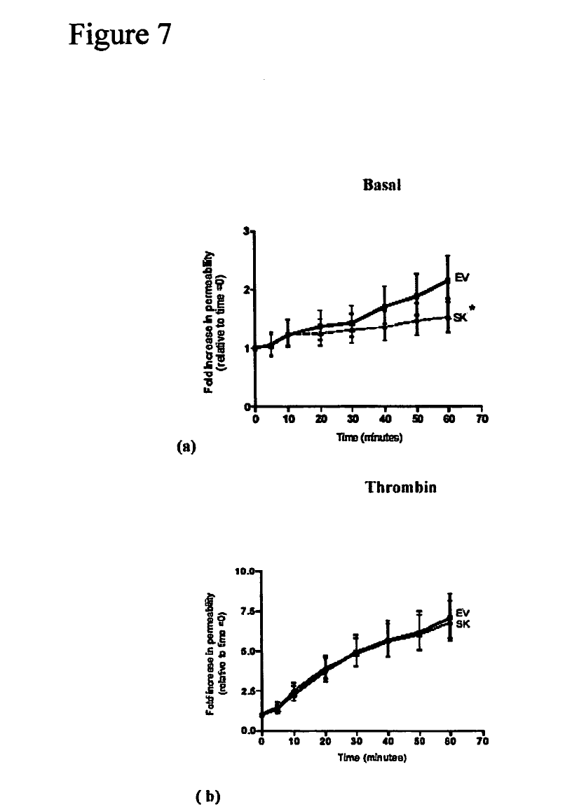

PECAM-1 is also involved in mediating cell-cell interactions important for control of junctional permeability. Consistent with an increase in PECAM-1 expression and a decrease in the phosphorylation of PECAM-1, cells over-expressing SK showed less basal permeability than control cells (FIG. 7a), although they responded normally to the known stimulator of permeability, thrombin (FIG. 7b).

SK-induced Survival is Mediated by PECAM-1

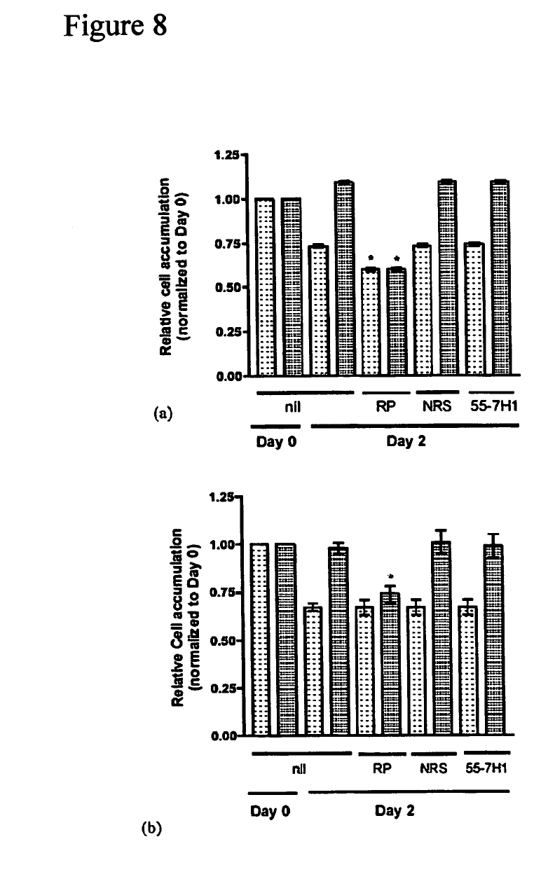

In light of the changes in PECAM-1 expression and regulation PECAM-1 was tested for responsibility for SK-induced endothelial cell survival both in suspension and in serum free conditions. Rabbit polyclonal anti-PECAM-1 antibody significantly reduced the survival of cells over-expressing SK both in serum free conditions and in suspension, while normal rabbit serum had no effect on either cells over-expressing SK or control cells (FIG. 8a,b). A murine monoclonal antibody directed to VE-cadherin (55-7H1) had no effect in reducing survival of cells over-expressing SK (p=0.61) or control cells (p=0.69). This indicates that SK-induced ability to survive in suspension is mediated by PECAM-1 and not through another junctional molecule VE cadherin.

SK Signals Through PECAM-1 to Activate the PI-3Kinase Pathway

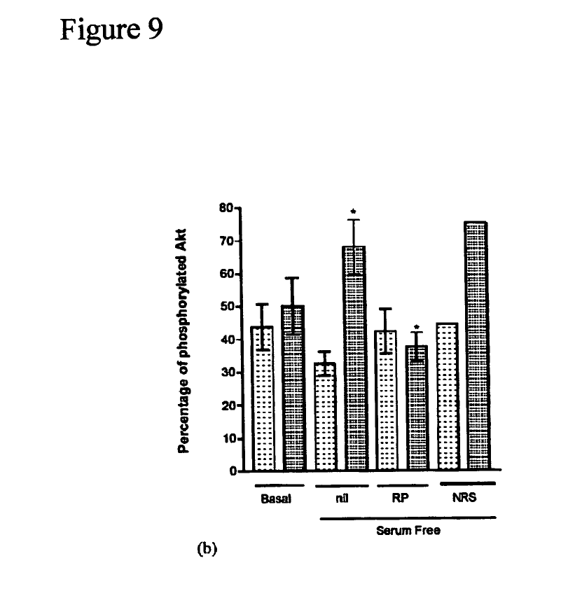

Total Akt and active (phosphorylated Akt) were measured by Western blot under basal conditions, in response to serum deprivation for six hours. Results are shown in FIG. 9(a), with quantitation shown in FIG. 9(b). The SK-mediated activation of Akt pathway in response to serum deprivation is again demonstrated. Rabbit polyclonal anti-PECAM-1 antibody (but not normal rabbit serum) reduced to control levels, the stress-induced-increase in phosphorylation of Akt for cells over-expressing SK, but had no effect in control cells.

SK-mediated Cell Survival Is Not Mediated By SIP Acting on GPCR

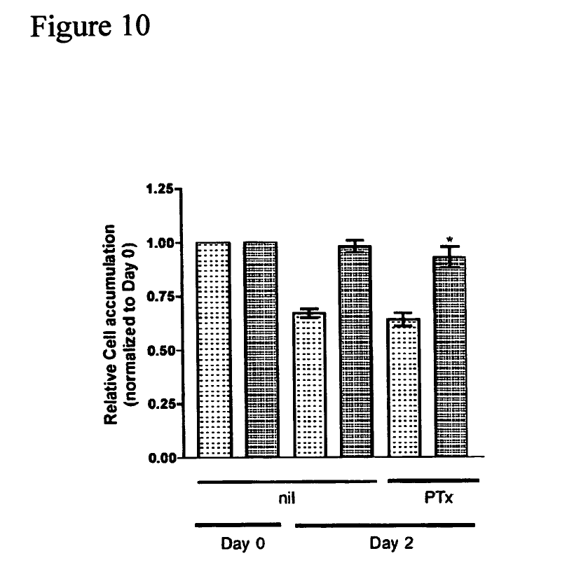

The downstream effector of SK, SIP, mediates cell survival through EDG receptors (a member of the pertussis toxin-sensitive G-protein coupled receptors). To determine whether it is possible that over-expression of SK leads to increased secretion of SIP that then acts exogenously, or whether SK itself is released with extracellular generation of S1P, the effect of inhibiting GPCR with pertussis toxin on cell survival was examined. SK-mediated cell survival was not inhibited in the presence of pertussis toxin (FIG. 10), consistent with an intracellular site of action of S1P. Exogenously added S1P had no effect on either the level of PECAM-1 expression or its phosphorylation status (data not shown), further suggesting that EDG activation is not involved in the PECAM-1-mediated changes in cell survival.

### EXAMPLE 2

### Sphingosine Kinase as a Novel Target for Modulation of Inflammation and Angiogenesis Methods

HUVEC Culture

HUVEC were isolated and cultured as previously described (Litwin M, el al. (1997) supra), with medium supplemented with 50 μg/ml endothelial cell growth supplement (Collaborative Research, MA, USA) and 50 μg/ml heparin (Sigma, St Louis, Mo., USA).

Adenovirus Production and Generation of Transient Cell Lines

The AdEasy system was used to produce recombinant adenovirus carrying SK, G82D, or empty vector (EV) according to the Qbiogene Version 1.4 AdEasy™ Vector system manual (http:www.qbiogene.com/products/adenovirus/adeasy.shtml). 293 cells were cultured in Dulbecco's modified Eagle's medium (CSL Biosciences, Parkville, Australia). Virus was amplified in 293 cells and purified on a cesium chloride gradient with centrifugation. The viral titre was determined using the TCID₅₀ method according to the manufacturer's protocol. Transient transfection of HUVEC was achieved by infection with adenoviral preparations of SK or EV using equivalent plaque forming units (pfu)/cell) which yielded a similar level of GFP expression.

Retrovirus Production and Generation of Stable Cell Lines

FLAG-epitope tagged SK, G82D (Pitson S M, et al. (2000) supra) or no construct (EV) were cloned into vector PrufNeo (Zannettino A C, Rayner J R, Ashman L K, Gonda T J, Simmons P J. (1996) J Immunol; 156(2):611-620). Retroviral production was undertaken by calcium phosphate transfection of PrufNeo-SK, PrufNeo-G82D or Pruf Neo-EV into Bing cells. The retroviral supernatant was collected at 48 hours. Stable cell lines were generated by infecting HUVEC with retroviral supernatant, followed by selection with G418 (Promega, Madison, Wis., USA) at 48 hours. Over-expression of SK was confirmed with both Western blot and SK activity assay.

Western Blotting

SDS-polyacrylamide gel electrophoresis was performed as described (Laemmli U K. (1970) supra) on cell lysates using 12% acrylamide gels. Proteins were transferred to PVDF membranes, blocked in 5% low fat milk in PBS with 0.1% Tween20 for one hour, and incubated overnight at 4° C. with M2 anti-FLAG antibody (Sigma, St Louis, Mo., USA). The membrane was incubated with horseradish peroxidase-conjugated anti-mouse IgG (Pierce) and immunocomplexes were detected using enhanced chemiluminescence (Amersham Pharmacia Biotech). SK activity SK activity was determined as previously described (11). Briefly, D-erythro-sphingosine and [γ-³²P]ATP were used as substrates, which were incubated with whole cell lysates. The labeled lipids were extracted and resolved by TLC. The radioactive spots were quantified by the Phosphoimage system.

Fluorescence Activated Cell Sorting (FACS)

Flow cytometric analysis of cell surface expression of E-Selectin and VCAM-1 was performed as previously described (11) using 10 μg/ml mouse monoclonal primary antibodies to E-Selectin (49-1B11) or VCAM-1 (51-10C9) generated in our laboratory (Gamble J R, Khew-Goodall Y, Vadas M A. (1993) supra). Secondary antibodies used were anti-mouse fluorescein-isothiocyanate, or for GFP-expressing cells, goat anti-mouse IgG R-phycoerythrin conjugate (Southern Biotech Birmingham, Ala., USA). The median fluorescence intensity was determined using a Coulter Epics Profile XL flow cytometer.

Tube Formation in Matrigel

A 96 well microtitre tray was coated with Matrigel Basement Membrane Matrix (Beckton Dickinson Labware, Bedford, Mass., USA). Endothelial cells were prepared at a concentration of 3×10⁵ cells/ml in HUVE medium and 140 μl was added to each well. The cells were visualized at regular intervals by microscopy to observe tube formation.

Neutrophil Adhesion Assay

HUVEC were seeded into fibronectin-coated Lab-Tek slides at 3×10⁴ cells per well and incubated at 37° C. for 24 hours. The cells were washed and then neutrophils were added to each well at 1×10⁵ cells per well. The cells were incubated at 37° C. for 30 minutes, and then any non-adherent neutrophils were removed by washing three times. The endothelial cells were fixed with methanol. The number of adherent neutrophils in consecutive fields was determined by microscopy.

Statistical Analysis

The Student's t-Test was used for parametric data, and p values less than 0.05 were considered significant. Significance testing for ratios was performed by ANOVA style regression using Statistica Version 6.1 (Statsoft, Inc.). The outcome measurements were all log transformed which ensured the predicted values were always positive and enabled interpretation of the analysis as the median fold change relative to a chosen baseline. The majority of the analyses were performed by normal linear regression and the reported p-values were determined by the t test with appropriate degrees of freedom. Mean (a) effects, relative to a specified baseline, and their associated standard errors (s.e.) were determined by appropriate linear contrasts of the regression coefficients. For analyses of log transformed outcome data, approximate large sample 95% confidence intervals (CI) were obtained using the formula: μ±1.96*s.e. The median fold change (relative to the specified baseline) with approximate 95% CI, were then obtained by back-transformation (i.e. exponentiation).

Results

Over-expression of SK Increases SK Activity

To determine the effect on endothelial cell function of over-expression of SK, HUVEC were infected with either retrovirus containing SK or adenovirus containing SK, at 1 pfu/cell. This level of adenovirus infection was selected since it resulted in similar levels of SK activity as TNFα-stimulation of endogenous SK in endothelial cells (12), and similar levels of SK activity as was achieved with retrovirus-mediated gene delivery.

Over-expression of SK Alters Adhesion Molecule Expression in HUVEC

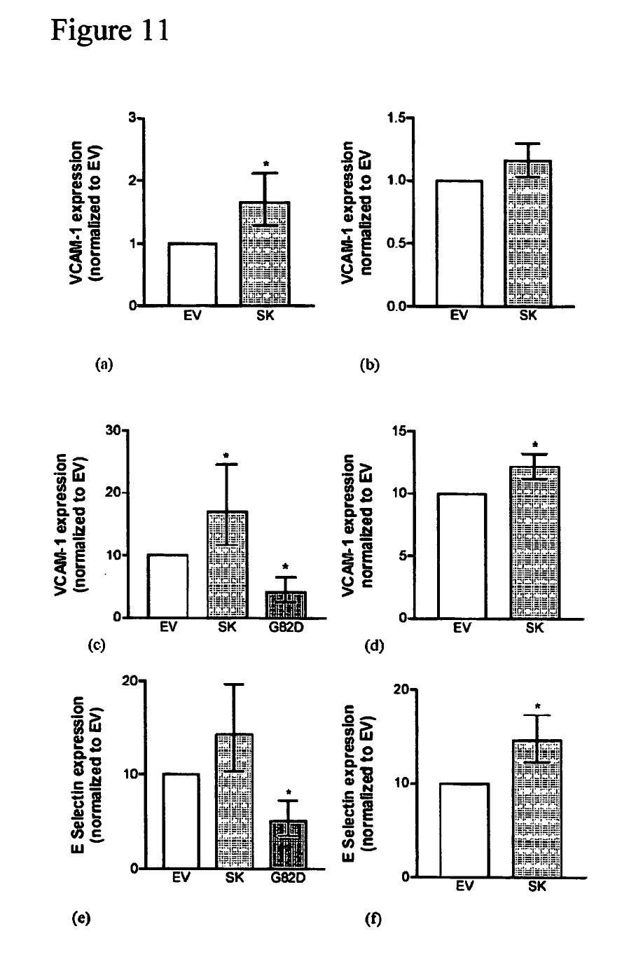

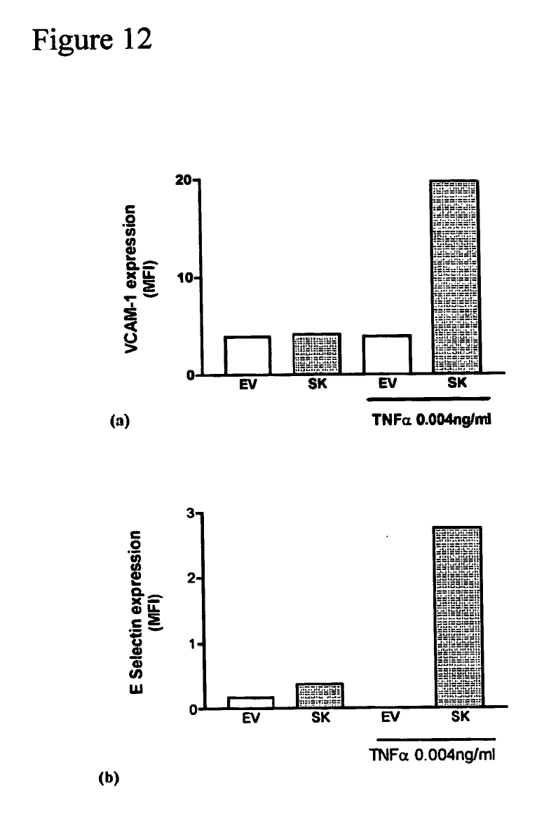

To determine whether over-expression of SK results in changes to the endogenous phenotype of endothelial cells, we investigated adhesion molecule expression was investigated on these infected cells. Retrovirus-mediated over-expression of SK up-regulated basal VCAM-1 expression (FIG. 11a). Adenoviral-mediated over-expression of SK resulted in a similar increase in VCAM-1 expression (p=0.052), as shown in FIG. 11b. This did not quite reach statistical significance, as the confidence intervals used were large sample confidence intervals, and were not adjusted for the degrees of freedom. Statistical significance was achieved by analysis of the data as a mean difference (p=0.04) or by the use of a non-parametric test. In contrast to VCAM-1, basal E Selectin expression was not altered in cells over-expressing SK generated by retroviral (n=4, p=0.44) or adenoviral (n=3, p=0.71)-mediated transfection. As over-expression of SK induced basal levels of VCAM-1 we next sought to determine whether these cells exhibited an altered response to stimulation with TNFα HUVEC overexpressing SK were stimulated with TNFα for four hours and adhesion molecule expression determined. Over-expression of SK achieved with either retroviral or adenoviral-mediated delivery significantly augmented the normal TNFα-induced up-regulation of VCAM-1 expression (FIG. 11c,d). Interestingly, cells over-expressing SK also showed an enhanced E Selectin response following stimulation with TNFα (FIG. 11e,f) even though basal E Selectin expression was not altered. Over-expression of dominant-negative SK (G82D) significantly inhibited the induction of VCAM-1 and E Selectin in response to TNFA compared with EV (FIG. 11c,e respectively). When cells were stimulated with subliminal doses of TNFα which failed to up-regulate VCAM-1 or E Selectin in the control, significant levels of both adhesion molecules were induced in cells over-expressing SK (FIG. 12a,b). The induction of VCAM-1 expression by TNFα in cells over-expressing SK was 4.42 (95% CI 1.51-12.94)-fold greater than EV cells (p<0.05) in three separate experiments. In two separate endothelial cell lines, the induction of E Selectin expression by TNFce was 1.7 and 3.1 -fold greater in cells over-expressing SK compared with EV cells. Induction of E Selectin on endothelial cells by TNFα peaks at 4-6 hours and declines to near basal levels by 18-24 hours (Gamble J R, Khew-Goodall Y, Vadas M A. (1993) supra; Gamble J R, Harlan J M, Klebanoff S J, Vadas M A. (1985) Proc. Natl. Acad. Sci. USA 82(24):8667-8671). To determine whether over-expression of SK altered this time course, cells infected with retrovirus carrying SK or EV were treated with TNFα at 0.5 ng/mL for 18 hours, and cell-surface expression of E Selectin was measured. Representative results are shown in Table 2. In four such lines there was a 2.01 (95% CI 1.14-3.53)-fold increase in E Selectin expression at 18 hours after stimulation with TNFα in cells over-expressing SK compared with EV cells (p=0.11). Over-expression of G82D resulted in a significant inhibition of this response (mean fold increase above control 0.44, 95% CI 0.25-0.92, p=0.014). Similar results were obtained with adenovirus-mediated gene transfer. Retroviral and adenoviral delivery of SK generated similar phenotypes in EC, that of enhanced expression of adhesion molecules and altered response to TNFα However the adenoviral system enabled large numbers of cells to be rapidly generated and therefore this method was used for future experiments.

Effects of Intracellular Over-expression of SK Are Not Mediated Through SIP Receptors

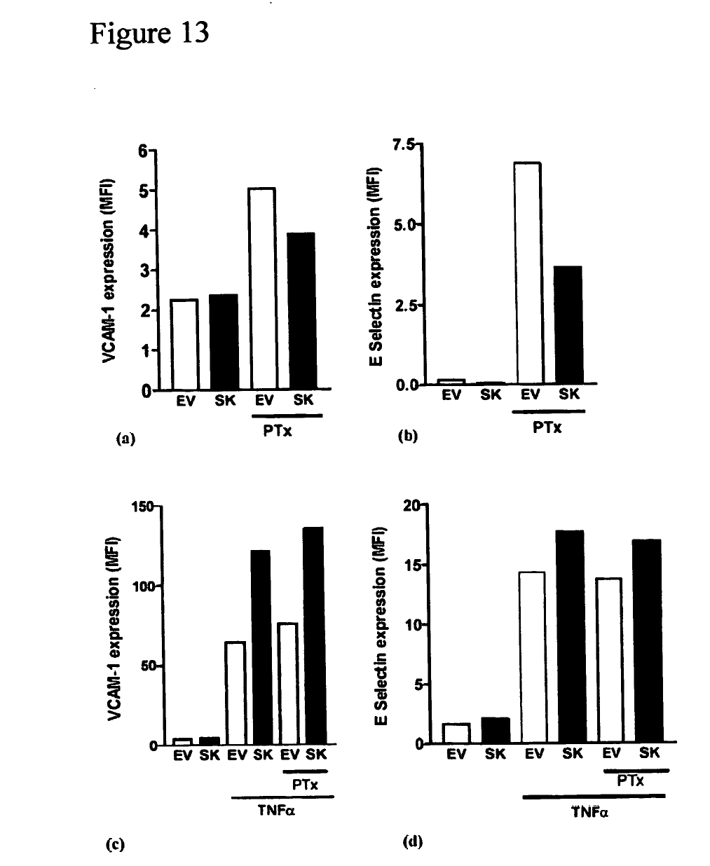

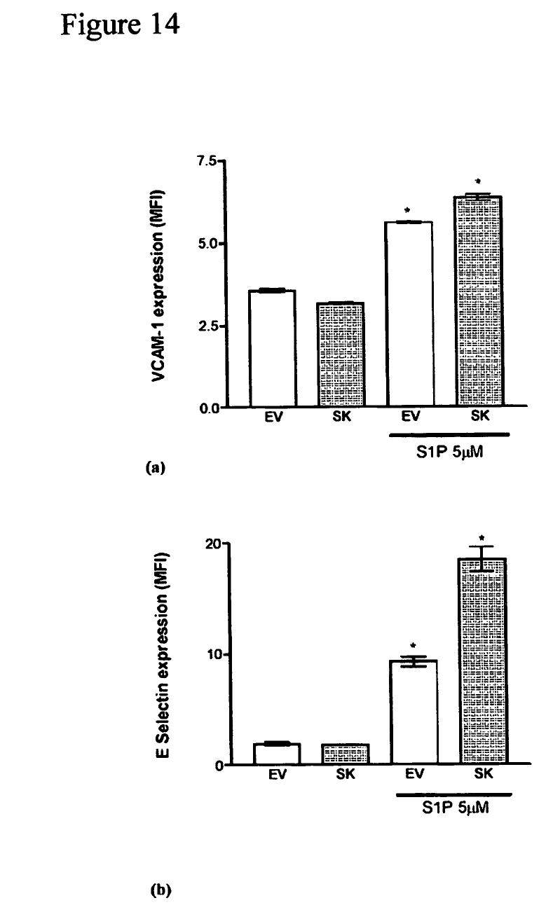

The EDG receptor which is responsible for SIP-induced up-regulation of adhesion molecules is known to be pertussis toxin sensitive, consistent with it being a G protein-coupled receptor. To investigate whether the results could be explained by secretion of SlP acting back on the EDG receptor, cells were treated with pertussis toxin (50 ng/ml) and adhesion molecule expression measured. Pertussis toxin did not inhibit basal or TNFα-induced VCAM-1 or E Selectin expression in either cells over-expressing SK or EV (FIG. 13). In actual fact, there was an enhancement of adhesion molecule expression seen with pertussis toxin treatment using two separate endothelial cell isolates. To determine whether the augmentation of the TNFα-induced adhesion molecule response in cells over-expressing SK was due to SIP acting on EDG receptors, cells were pre-treated with pertussis toxin (50 ng/ml) for 18 hours and then stimulated with TNFα (0.5 ng/ml) for four hours in the presence of pertussis toxin. Adhesion molecule expression was measured in these cells. In two separate endothelial cell lines, pre-treatment with pertussis toxin did not alter TNFα-induced VCAM-1 or E Selectin expression in control cells or cells overexpressing SK (FIG. 13c,d). To further delineate intracellular versus EDG receptor-mediated effects of SIP we stimulated cells over-expressing SK with exogenous SIP (5 μM) for four hours. Both cells overexpressing SK and EV cells responded to exogenously added SIP by up-regulation of VCAM-1 and E Selectin expression, suggesting the EDG receptor was still operating normally as shown in FIG. 14. It was of interest that the E Selectin response to SIP stimulation was 1.75 (95% CI 1.4-2.18)-fold greater in cells over-expressing SK compared with control (p<0.001), suggesting that over-expression of SK sensitizes the cells to SIP. Although pertussis toxin failed to inhibit the augmented TNFα-induced adhesion molecule response in cells over-expressing SK, pretreatment with pertussis toxin inhibited the response to exogenous stimulation with SIP in both cells over-expressing SK and EV, providing further support for an intracellular role for SK.

SK Enhances Neutrophil Adhesion to Endothelial Cells

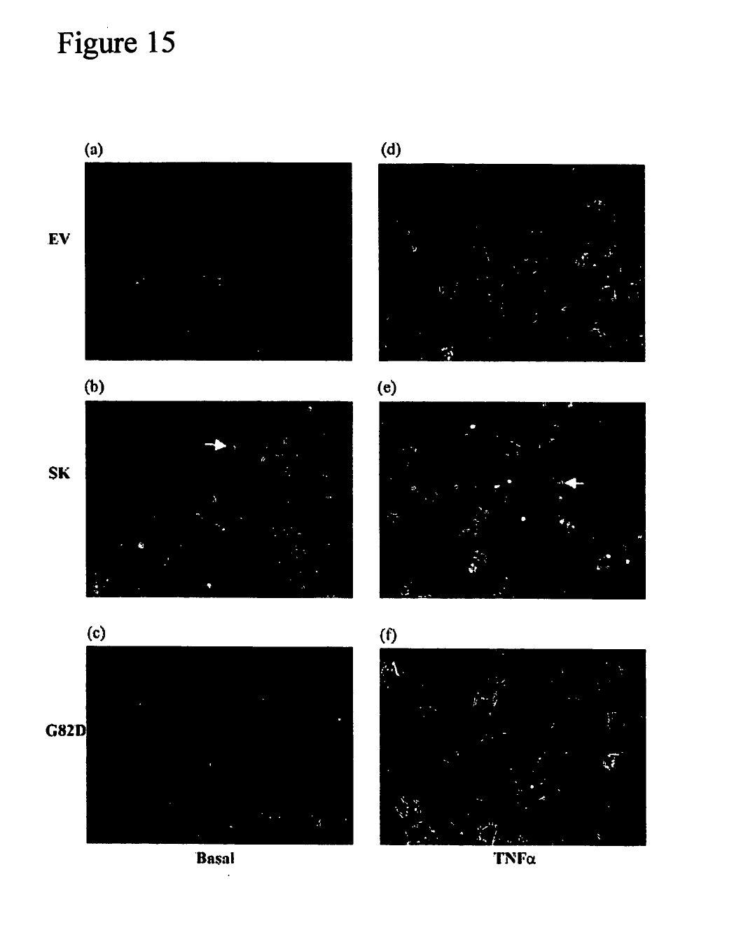

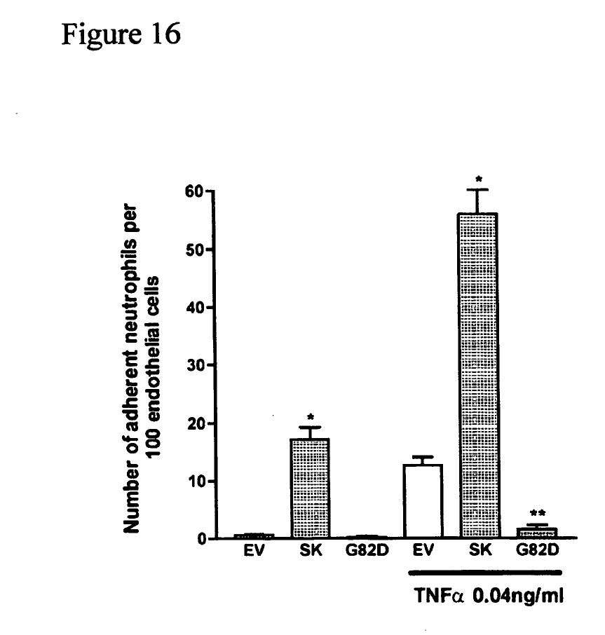

To determine whether the alteration in adhesion molecule expression resulting from intracellular over-expression of SK had functional consequences, neutrophil adhesion to endothelial cells was measured. In the basal state, cells over-expressing SK showed significant neutrophil adhesion, which is in contrast with control cells which did not bind neutrophils (FIG. 15a,b). Stimulation of endothelial cells with a low dose of TNFα (0.04 ng/ml) resulted in minimal neutrophil adhesion in control cells (FIG. 15d), but significantly greater adhesion to cells over-expressing SK (FIG. 15e). Consistent with a role for SK in mediating PMN adhesion, endothelial cells over-expressing the dominant-negative SK, G82D, inhibited PMN adhesion in response to stimulation with TNFα (FIG. 15c,f). Quantitation of the number of neutrophils attached per 100 endothelial cells is shown in FIG. 16.

SK Promotes Tube Formation

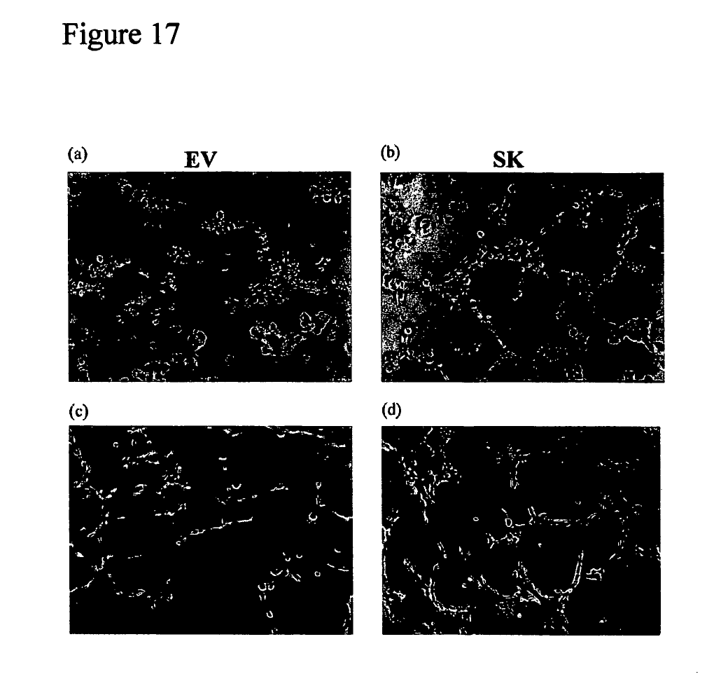

The ability of endothelial cells to arrange into capillary like networks (tubes) is an vitro correlate of angiogenesis and angiogenesis is a characteristic feature of many chronic inflammatory diseases. Therefore it was sought to determine whether SK over-expression also enhances the ability of endothelial cells to form tubes. Endothelial cells were plated onto the complex basement membrane matrix, Matrigel. Equivalent numbers of cells over-expressing SK and EV were seeded, and cells were visualized as single cell populations. Within 15 minutes of seeding, cells over-expressing SK had already commenced realignment whereas the EV cells remained disorganized. By 30 minutes cells over-expressing SK showed greater evidence of tube alignment compared with EV cells (FIG. 17a,b). By one hour tube formation by cells over-expressing SK was highly developed compared with EV cells (FIG. 17c,d). By 18 hours, a time where tube formation was complete, both cells over-expressing SK and EV cells showed a similar pattern of tube formation. These results suggest that over-expression of SK stimulates the rate of tube formation.

TABLE 2
E Selectin expression (MFI)

| | Basal | TNFα 4 hr | TNFα 18 hr |
|---|---|---|---|
| EV | 0.035 | 45.0 | 0.66 |
| SK | 0.05 | 74.8 | 2.37 |

Table 2 shows basal and stimulated (TNFα 0.5 ng/ml for 4 or 18 hours) E Selectin expression as indicated by the median fluorescence intensity (MFI) in cells infected with retrovirus carrying SK or control (EV). The table shows the results from a single endothelial cell line which is representative of four separate endothelial cell lines tested.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Bunin B A, et al. (1994) Proc. Natl. Acad. Sci. USA, 91:4708-4712.

DeWitt S H, et al. (1993) Proc. Natl. Acad. Sci. USA, 90:6909-6913

Gamble J R, Khew-Goodall Y, Vadas M A. (1993) J Immunol; 150(10):4494-4503

Gamble et al, (1993) J Cell Biol 121:931-945

Laernmli U K. (1970) Nature; 227(259):680-685

Litwin M, Clark K, Noack L, Furze J, Berndt M, Albelda S et al. (1997) J Cell Biol 139(1):219-228

Pitson S M, Moretti P A, Zebol J R, Xia P, Gamble J R, Vadas M A et al. (2000) J Biol Chem; 275(43):33945-33950

Xia P, Gamble J R, Rye K A, Wang L, Hii C S, Cockerill P et al. (1998) Proc Natl Acad Sci USA; 95(24):14196-14201

Zannettino A C, Rayner J R, Ashman L K, Gonda T J, Simmons P J. (1996) J Immunol; 156(2):611-620

(25) Gamble J R, Harlan J M, Klebanoff S J, Vadas M A. (1985) Proc. Natl. Acad. Sci. USA 82(24):8667-8671.

1. A method of modulating one or more mammalian endothelial cell functional characteristics, said method comprising modulating the functional level of sphingosine kinase wherein inducing over-expression of said sphingosine kinase level modulates one or more of the functional characteristics of said endothelial cell.

2. A method of modulating one or more endothelial cell functional characteristics in a mammal, said method comprising modulating the functional level of sphingosine kinase wherein inducing over-expression of said sphingosine kinase level modulates one or more of the functional characteristics of said endothelial cell.

3. A method for the treatment and/or prophylaxis of a condition characterised by aberrant or otherwise unwanted endothelial cell functioning in a mammal, said method comprising modulating the functional level of sphingosine kinase in said mammal wherein inducing over-expression of said sphingosine kinase level modulates one or more functional characteristics of said endothelial cells.

4. The method according to any one of claims 1-3 wherein said endothelial cell is a vascular endothelial cell.

5. The method according to claim 4 wherein said endothelial cell functional characteristic is up-regulatable by sphingosine kinase over-expression and said characteristic is one or more of viability, proliferation, differentiation, cell surface molecule expression, cytokine responsiveness or enhanced proliferation or viability.

6. The method according to claim 5 wherein said cell surface molecule is an adhesion molecule.

7. The method according to claim 5 or 6 wherein said functional characteristic is up-regulated.

8. The method according to claim 4 wherein said endothelial cell functional characteristic is up-regulatable by sphingosine kinase over-expression and said characteristic is the induction of a pro-inflammatory phenotype.

9. The method according to claim 8 wherein said pro-inflammatory phenotype is down-regulated.

10. The method according to claim 4 wherein said endothelial cell functional characteristic is up-regulatable by sphingosine kinase over-expression and said characteristic is the induction of an angiogenic phenotype.

11. The method according to claim 10 wherein said angiogenic phenotype is up-regulated.

12. The method according to claim 10 wherein said angiogenic phenotype is down-regulated.

13. The method according to claim 4 wherein said endothelial cell functional characteristic is up-regulatable by sphingosine kinase over-expression and said characteristic is maintenance of the CD34⁺ endothelial cell progenitor phenotype.

14. The method according to claim 13 wherein said CD34⁺ progenitor phenotype is maintained.

15. The method according to claim 3 wherein said condition is vascular engraftment, wound repair, tissue or organ transplantation or the repair of devascularised tissue and said modulated endothelial cell functional characteristic is one or more of enhanced endothelial cell proliferation, enhanced endothelial cell viability or maintenance of the CD34⁺ endothelial cell progenitor phenotype.

16. The method according to claim 3 wherein said condition is an inflammatory condition and said modulated endothelial cell functional characteristic is down-regulation of one or more of an endothelial cell inflammatory or angiogenic phenotype.

17. The method according to claim 16 wherein said condition is rheumatoid arthritis.

18. The method according to claim 3 wherein said condition is characterised by unwanted angiogenesis and said modulated endothelial cell functional characteristic is down-regulation of an endothelial cell angiogenic phenotype.

19. The method according to claim 18 wherein said condition is a tumour.

20. The method according to any one of claims 1-8, 10-11 or 13-15 wherein said modulation is up-regulation of sphingosine kinase levels and said up-regulation is achieved by introducing into said endothelial cell a nucleic acid molecule encoding sphingosine kinase or functional equivalent, derivative or homologue thereof or the sphingosine kinase expression product or functional derivative, homologue, analogue, equivalent or mimetic thereof.

21. The method according to any one of claims 1-19 wherein said modulation is achieved by contacting said endothelial cell with a proteinaceous or non-proteinaceous molecule which modulates transcriptional and/or translational regulation of the sphingosine kinase gene.

22. The method according to any one of claims 1-8, 10-11 or 13-15 wherein said modulation is up-regulation of sphingosine kinase levels and said up-regulation is achieved by contacting said endothelial cell with a proteinaceous or non-proteinaceous molecule which functions as an agonist of the sphingosine kinase expression product.

23. The method according to any one of claims 1-6, 8-10, 12-13 or 16-19 wherein said modulation is down-regulation of sphingosine kinase levels and said down-regulation is achieved by contacting said endothelial cell with a proteinaceous or non-proteinaceous molecule which functions as an antagonist to the sphingosine kinase expression product.

24. The method according to claim 23 wherein said molecule is a mutant sphingosine kinase which mutant is characterised by substitution of the glycine residue at position 82 to aspartate.

25. The method according to any one of claims 1 or 2 wherein said endothelial cell activity is modulated in vivo.

26. The method according to any one of claims 1 or 2 wherein said endothelial cell activity is modulated in vitro.

27. Use of an agent capable of modulating the functional level of sphingosine kinase in the manufacture of a medicament for the modulation of one or more endothelial cell functional characteristics in a mammal wherein inducing over-expression of said sphingosine kinase level modulates one or more of the functional characteristics of said endothelial cells.

28. Use according to claim 27 wherein said agent is a proteinaceous or non-proteinaceous molecule which modulates transcriptional and/or translational regulation of the sphingosine kinase gene, functions as an agonist of sphingosine kinase activity or functions as an antagonist of sphingosine kinase activity.

29. Use of sphingosine kinase or a nucleic acid encoding sphingosine kinase in the manufacture of a medicament for the modulation of one or more endothelial cell functional characteristics in a mammal wherein inducing over-expression of said sphingosine kinase level modulates one or more of the functional characteristics of said endothelial cells.

30. Use according to any one of claims 27-29 wherein said endothelial cell is a vascular endothelial cell.

31. Use according to claim 30 wherein said endothelial cell functional characteristic is up-regulatable by sphingosine kinase over-expression and said characteristic is one or more of viability, proliferation, differentiation, cell surface molecule expression, cytokine responsiveness or enhanced proliferation or viability.

32. Use according to claim 31 wherein said cell surface molecule is an adhesion molecule.

33. Use according to claim 30 wherein said endothelial cell functional characteristic is up-regulatable by sphingosine kinase over-expression and said characteristic is the induction of a pro-inflammatory phenotype.

34. Use according to claim 30 wherein said endothelial cell functional characteristic is up-regulatable by sphingosine kinase over-expression and said characteristic is the induction of an angiogenic phenotype.

35. Use according to claim 30 wherein said endothelial cell functional characteristic is up-regulatable by sphingosine kinase over-expression and said characteristic is maintenance of the CD34⁺ endothelial cell progenitor phenotype.

36. Use according to claim 35 wherein said CD34⁺ progenitor phenotype is maintained.

37. Use according to any one of claims 27-36 wherein said medicament is used to treat a condition characterised by aberrant or otherwise unwanted endothelial cell functioning.

38. Use according to claim 37 wherein said condition is vascular engraftment, wound repair, tissue or organ transplantation or the repair of devascularised tissue and said modulated endothelial cell functional characteristic is one or more of enhanced endothelial cell proliferation, enhanced endothelial cell viability or maintenance of the CD34⁺ endothelial cell progenitor phenotype.

39. Use according to claim 37 wherein said condition is an inflammatory condition and said modulated endothelial cell functional characteristic is down-regulation of one or more of an endothelial cell inflammatory or angiogenic phenotype.

40. Use according to claim 39 wherein said condition is rheumatoid arthritis.

41. Use according to claim 37 wherein said condition is characterised by unwanted angiogenesis and said modulated endothelial cell functional characteristic is down-regulation of an endothelial cell angiogenic phenotype.

42. Use according to claim 41 wherein said condition is a tumour.

43. A pharmaceutical composition comprising modulatory agent and one or more pharmaceutically acceptable carriers and/or diluents when used in the method of any one of claims 1-26.
